(19) United States
(12) Patent Application Publication
(10) Pub. No.: US 2024/0175005 A1
(43) Pub. Date: May 30, 2024

(54) PURIFICATION AND RECYCLING OF mRNA NUCLEOTIDE CAPS

(71) Applicant: ModernaTX, Inc., Cambridge, MA (US)

(72) Inventors: Jennifer Beaudoin, Littleton, MA (US); Lance Dieter, Acton, MA (US); Kaelyn Vik, Brig (CH)

(21) Appl. No.: 18/284,177

(22) PCT Filed: Mar. 31, 2022
(86) PCT No.: PCT/US2022/022836

§ 371 Date: Sep. 26, 2023

Related U.S. Application Data
(60) Provisional application No. 63/169,115, filed on Mar. 31, 2021.
(60) Provisional application No. 63/320,128, filed on Mar. 15, 2022.

(51) Int. Cl. C12N 15/10; B01D 15/32; B01D 15/36; B01D 61/14; B01D 63/10; B01D 71/10; B01D 71/56

(52) U.S. Cl. CPC C12N 15/1017; B01D 15/325; B01D 15/363; B01D 61/145; B01D 63/10; B01D 71/10; B01D 71/56; C12N 15/101; B01D 2315/10

(57) ABSTRACT

This disclosure relates to purification and recycling nucleotide messenger RNA (mRNA) caps from the preparation of mRNA, which comprises isolating and purifying excess nucleotide mRNA caps from mRNA synthesis.

### TECHNICAL FIELD

This disclosure relates to purification and recycling nucleotide messenger RNA (mRNA) caps from the preparation of mRNA, which comprises isolating and purifying excess nucleotide mRNA caps from mRNA synthesis.

### BACKGROUND

There is great interest in the field of therapeutics to be able to generate mRNA efficiently. For example, mRNA can be encapsulated in lipid nanoparticles and delivered to a subject for treatment or prevention of various diseases or conditions. The five-prime cap (5′ cap) is added to the first nucleotide in the transcript during transcription, and this process of mRNA capping is important in protecting the transcript from being broken down. mRNA production costs can be relatively high which in part is due to the cost of preparing the nucleotide mRNA caps. Further, the production of mRNA typically requires the using excess of the nucleotide mRNA caps. There remains needs to be able to recapture and recycle the excess nucleotide caps employed in the production of mRNA, which would make the overall process of making mRNA more economical especially on large-scale production. This application addresses these needs.

### BRIEF SUMMARY

Provided herein are methods of recycling mRNA nucleotide caps or salts thereof, from an mRNA preparation. In one aspect, the method comprises:
collecting and combining one or more mixtures comprising the mRNA nucleotide cap, or a salt thereof, and one or more contaminants; and
removing the contaminants from the combined mixtures.

In one aspect, the method provided herein includes removing the contaminants from the combined mixtures, which can include removing macromolecules and proteins from the combined mixture comprising the mRNA nucleotide cap, or a salt thereof, to provide a first mixture.

In one aspect, the removal of the contaminants can further include concentrating and de-salting the first mixture to provide a second mixture.

In one aspect, the removal of contaminants can further include removing nucleotide triphosphates (NTPs) and ion exchanging from the second mixture to provide a third mixture.

In one aspect, the removal of contaminants further includes concentrating and de-salting the third mixture to provide a fourth mixture.

In one aspect, the removal of the contaminants can further include filtering, and adjusting the concentration and pH of the fourth mixture.

In one aspect, the removal of the contaminants from the combined mixtures comprises:
removing macromolecules and proteins from the combined mixture comprising the mRNA nucleotide cap, or a salt thereof, to provide a first mixture;
concentrating and de-salting the first mixture to provide a second mixture;
removing nucleotide triphosphates (NTPs) and ion exchanging from the second mixture to provide a third mixture; and
concentrating and de-salting the third mixture to provide a fourth mixture.

In one aspect, the removal of the contaminants from the combined mixture can include:
removing macromolecules and proteins from the combined mixture comprising the mRNA nucleotide cap, or a salt thereof, to provide a first mixture;
concentrating and de-salting the first mixture to provide a second mixture;
removing nucleotide triphosphates (NTPs) and ion exchanging from the second mixture to provide a third mixture;
concentrating and de-salting the third mixture to provide a fourth mixture; and
filtering, and adjusting the concentration and pH of the fourth mixture.

In one aspect, the mRNA nucleotide cap, or a salt thereof, prepared by a method described herein has a purity greater than about 90%.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting.

### BRIEF DESCRIPTION OF THE DRAWINGS

FIG. 1 shows an exemplary schematic of the recycling purification process for Compound A.

FIG. 2 shows an exemplary schematic of an alternative recycling purification process for Compound A.

FIG. 3 shows a LCMS of a mixture comprising the mRNA nucleotide cap collected from an in vitro transcription preparation and LCMS of recycled Compound A.

FIG. 4 shows a H¹ NMR of recycled Compound A N,N-dimethyloctylammonium (DMOA) salt, and H¹ NMR of recycled Compound A NH₄⁺ salt.

FIG. 5 shows an exemplary schematic of the recycling purification processes for Compound G.

FIG. 6 shows a LCMS of retentate containing recycled Compound G after filtration using tangential flow filtration (TFF) with a 2 kDa filter with a starting purity of 50% and LCMS of pooled fractions containing recycled Compound G after anion exchange chromatography.

FIG. 7 shows an exemplary schematic of the ion exchange chromatography system.

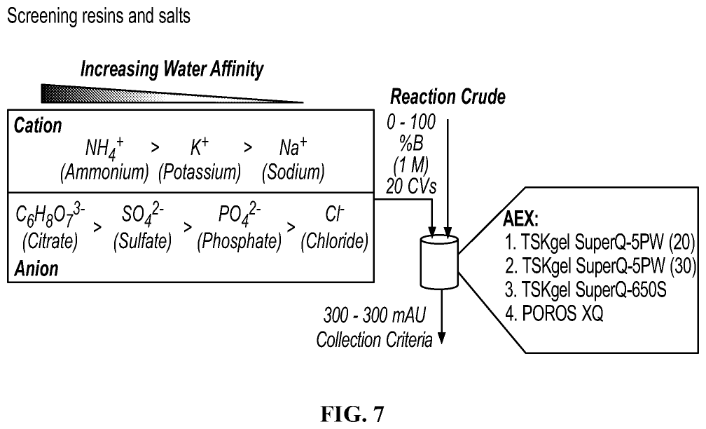

FIG. 8 shows an exemplary schematic of an alternative recycling purification.

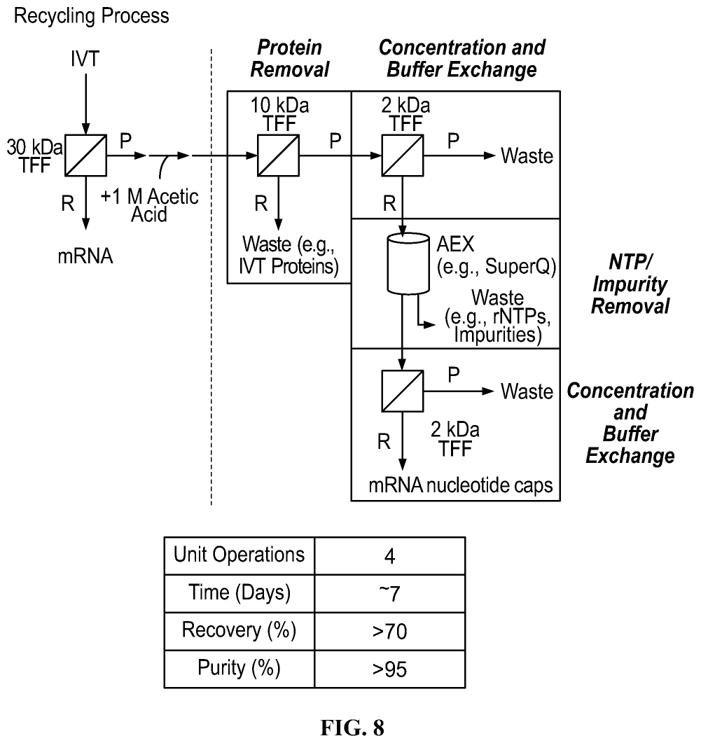

### DETAILED DESCRIPTION

Provided herein are methods of recycling mRNA nucleotide caps or salts thereof mRNA consists of an open reading frame (ORF) flanked by the 5′- and 3′-untranslated region (5′UTR, 3′UTR), a poly-adenosine monophosphate tail (polyA) and an inverted N7-methylguanosine containing cap structure. The cap-structure is a crucial feature of all eukaryotic mRNAs. It is recognized by the ribosomal complex through the eukaryotic initiation factor 4E (eIF4E). mRNAs lacking the 5′-cap terminus are not recognized by the translational machinery and are incapable of producing the target protein (see, e.g., C. Aitken, et al. “A mechanistic overview of translation initiation in eukaryotes”, Nature Structural and Molecular Biology, vol. 16, no. 6, 568-576, 2012). The crude mRNA produced during the transcription process (“primary transcript”) is terminated by a 5′-triphosphate, which is converted to the respective 5′-diphosphate by the action of the enzyme RNA-triphosphatase. Then a guanylyl-transferase attaches the terminal inverted guanosine monophosphate to the 5′-terminus, and an N7MTase-mediated N7-methylation of the terminal, inverted guanosine, completes the capping process.

Endogenous mRNA molecules can be 5′-end capped generating a 5′-ppp-5′-triphosphate linkage between a terminal guanosine cap residue and the 5′-terminal transcribed sense nucleotide of the mRNA molecule. This 5′-guanylate cap can then be methylated to generate an N7-methyl-guanylate residue. The ribose sugars of the terminal and/or anteterminal transcribed nucleotides of the 5′ end of the mRNA can optionally also be 2′-O-methylated.

Multiple distinct 5′-cap structures can be used to generate the 5′-cap of a nucleic acid molecule, such as a polynucleotide that functions as an mRNA molecule. Cap analogs differ from natural (i.e., endogenous, wild-type or physiological) 5′-caps in their chemical structure, while retaining cap function. Cap analogs can be chemically (i.e., non-enzymatically) or enzymatically synthesized.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m⁷G-3′mppp-G; which can equivalently be designated 3′ O-Me-m⁷G(5′)ppp(5′)G). The 3′-O atom of the other, unmodified, guanine becomes linked to the 5′-terminal nucleotide of the capped polynucleotide. The N7- and 3′-O-methlyated guanine provides the terminal moiety of the capped polynucleotide.

Another exemplary cap is mCAP, which is similar to ARCA but has a 2′ O-methyl group on guanosine (i.e., N7,2′-O-dimethyl-guanosine-5′-triphosphate-5′-guanosine, m⁷Gm-ppp-G). Another exemplary cap is m⁷G-ppp-Gm-A (i.e., N7,guanosine-5′-triphosphate-2′-O-dimethyl-guanosine-adenosine).

The cap can be modified at different phosphate positions with a boranophosphate group or a phosphoroselenoate group such as the caps described in U.S. Pat. No. 8,519,110, the contents of which are herein incorporated by reference in its entirety.

In some embodiments, the cap is a N7-(4-chlorophenoxyethyl) substituted form of a cap analog known in the art and/or described herein. Non-limiting examples of a N7-(4-chlorophenoxyethyl) substituted form of a cap analog include a N7-(4-chlorophenoxyethyl)-G(5′)ppp(5′)G and a N7-(4-chlorophenoxyethyl)-m^{3′-O}G(5′)ppp(5′)G cap analog (See, e.g., the various cap analogs and the methods of synthesizing cap analogs described in Kore et al. Bioorganic & Medicinal Chemistry 2013 21:4570-4574; the contents of which are herein incorporated by reference in its entirety). A cap analog can be a 4-chloro/bromophenoxyethyl analog.

5′ terminal caps can include endogenous caps or cap analogs. A 5′ terminal cap can comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

Other examples of mRNA nucleotide caps are described in US 20190211368, U.S. Ser. No. 10/563,195, U.S. Ser. No. 10/428,106, or U.S. Ser. No. 10/570,388, each of which is incorporated herein by reference.

mRNA nucleotide caps are typically used in excess in the preparation of mRNA. The methods described herein provide procedures for collecting and combining the mixtures from, e.g., mRNA preparation, that contain unused nucleotide caps, and removing the contaminants to provide purified nucleotide caps. The recycle methods described herein are efficient and can recapture the nucleotide caps in high yields, e.g., greater than about 80%. In some instances, the yields can be greater than 90%. The purity of the recycled nucleotide can be greater than 90%, greater than 98%, or greater than about 99%. In some instances, the purity of the recycled mRNA nucleotide is greater than 99.5%. Further, mRNAs generated using the recycled mRNA nucleotide caps described herein have substantially the same integrity (e.g., similar percent of tail and cap) as mRNA having nucleotide caps prepared from de novo synthesis.

The method provided herein of recycling mRNA nucleotide cap, or a salt thereof, comprises:
collecting and combining one or more mixtures comprising the mRNA nucleotide cap, or a salt thereof, from an mRNA preparation, and one or more contaminants; and
removing the contaminants from the combined mixtures.

The methods disclosed herein include recycling of the mRNA nucleotide caps or a salt thereof.

In some embodiments, the mRNA nucleotide cap, or a salt thereof, is:

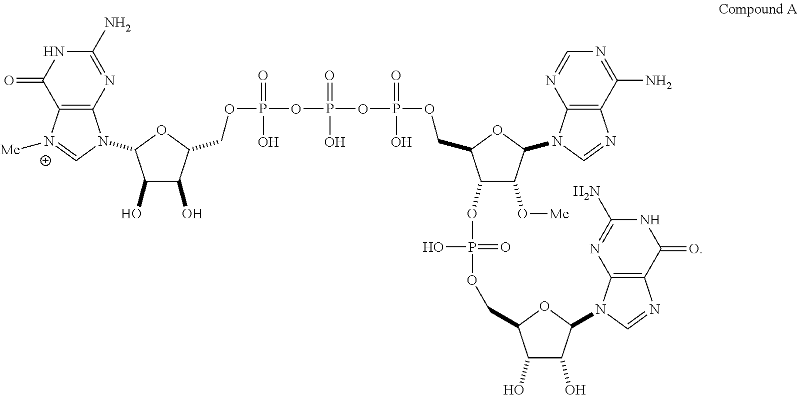

In some embodiments, the mRNA nucleotide cap, or a salt thereof, is:

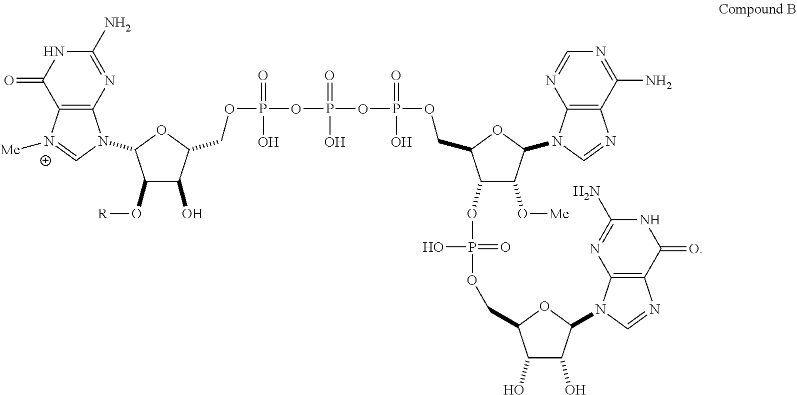

In some embodiments, the mRNA nucleotide cap, or a salt thereof, is:

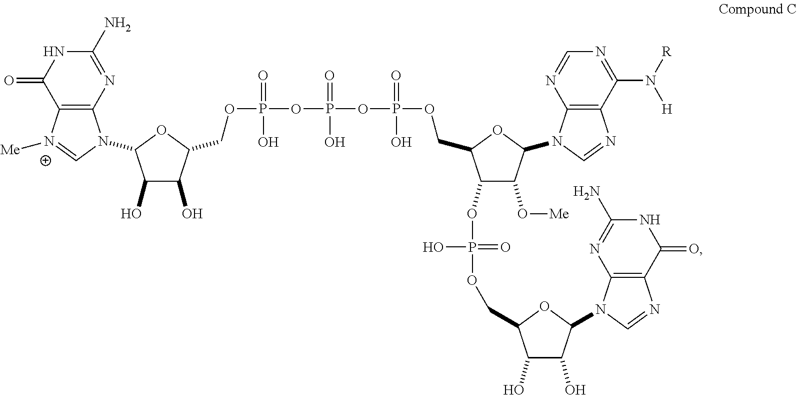

wherein R is an alkyl (e.g., C₁-C₆ alkyl). In some embodiments, R is a methyl group (e.g., C₁ alkyl). In some embodiments, R is an ethyl group (e.g., C₂ alkyl).

In some embodiments, the mRNA nucleotide cap, or a salt thereof, is:

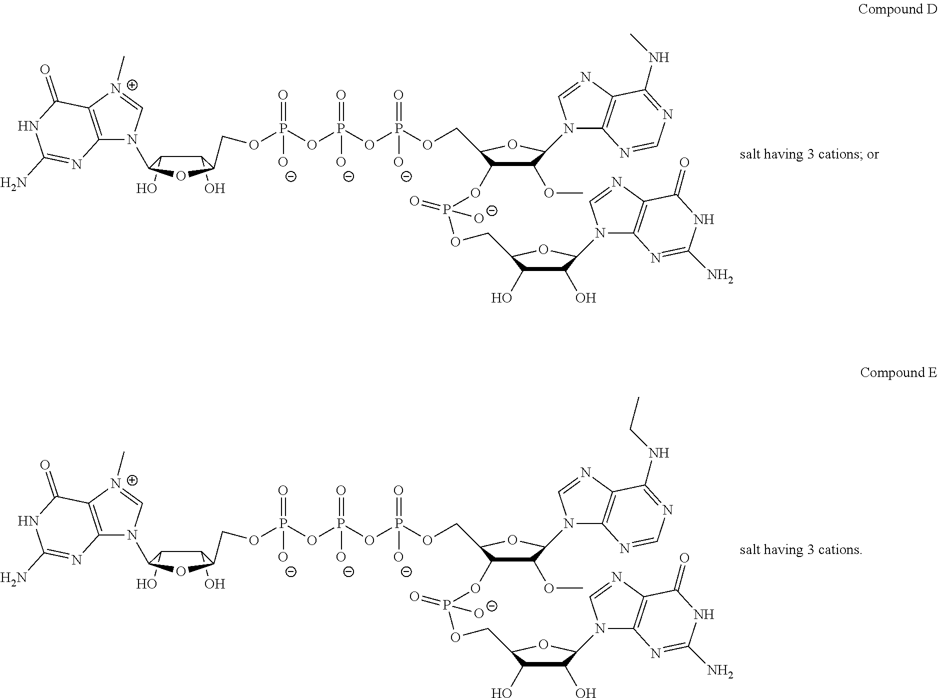

In some embodiments, the mRNA nucleotide cap, or a salt thereof, is:

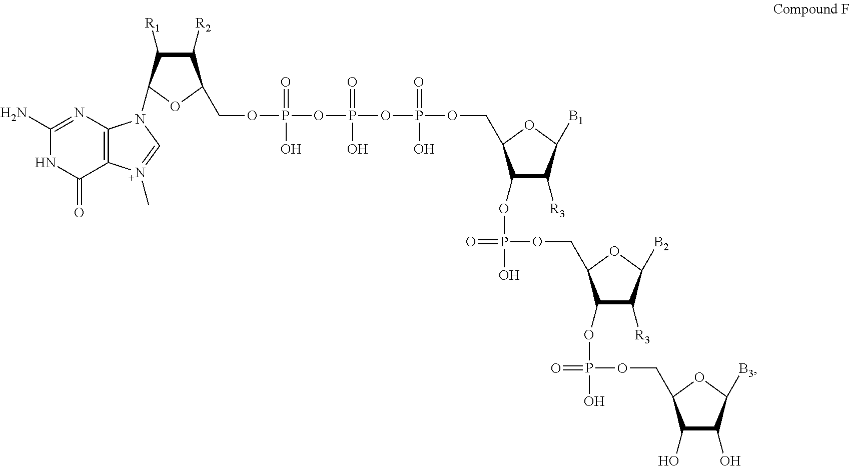

wherein:
B₁, B₂, and B₃ are independently a natural, a modified, or an unnatural nucleoside based; and R₁, R₂, R₃, and R₄ are independently OH or O-methyl. In some embodiments, R₃ is O-methyl and R₄ is OH. In some embodiments, R₃ and R₄ are O-methyl. In some embodiments, R₄ is O-methyl. In some embodiments, R₁ is OH, R₂ is OH, R₃ is O-methyl, and R₄ is OH. In some embodiments, R₁ is OH, R₂ is OH, R₃ is O-methyl, and R₄ is O-methyl. In some embodiments, at least one of R₁ and R₂ is O-methyl, R₃ is O-methyl, and R₄ is OH. In some embodiments, at least one of R₁ and R₂ is O-methyl, R₃ is O-methyl, and R₄ is O-methyl.

In some embodiments, B₁, B₂, and B₃ are natural nucleoside bases. In some embodiments, at least one of B₁, B₂, and B₃ is a modified or unnatural base. In some embodiments, at least one of B₁, B₂, and B₃ is N6-methyladenine. In some embodiments, B₁ is adenine, cytosine, thymine, or uracil. In some embodiments, B₁ is adenine, B₂ is uracil, and B₃ is adenine. In some embodiments, R₁ and R₂ are OH, R₃ and R₄ are O-methyl, B₁ is adenine, B₂ is uracil, and B₃ is adenine.

In some embodiments, the mRNA nucleotide cap, or a salt thereof, is:

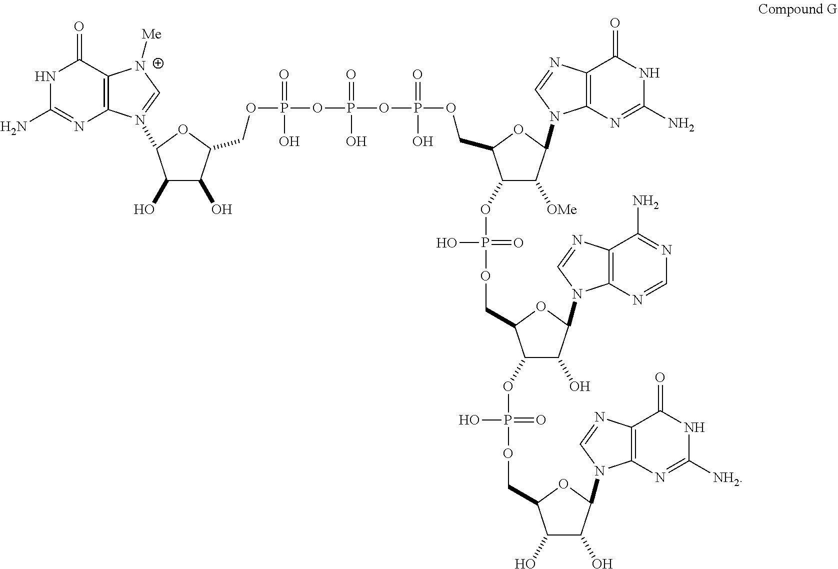

In some embodiments, the mRNA nucleotide cap, or a salt thereof, is:

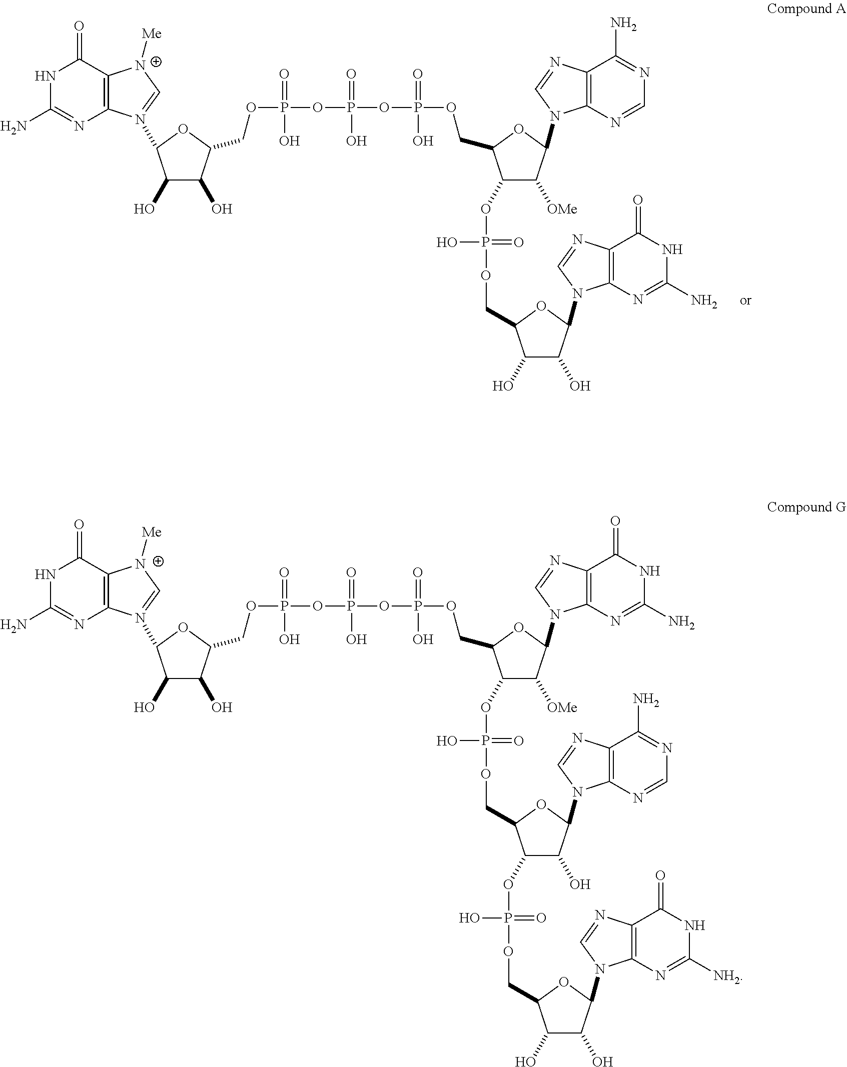

In some embodiments, the mRNA nucleotide cap, or a salt thereof, is:

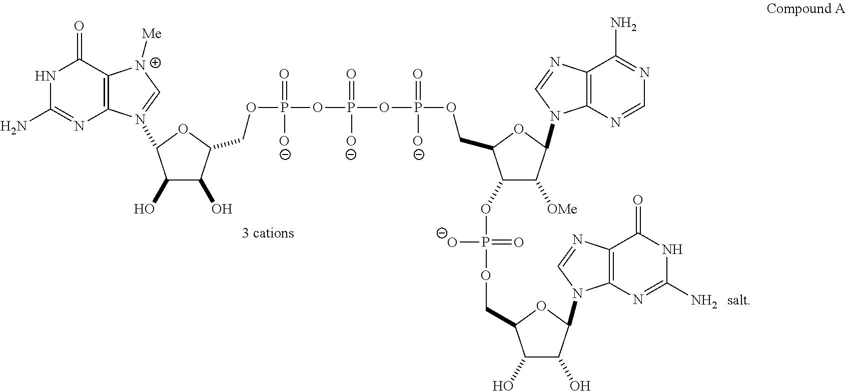

In some embodiments, the mRNA nucleotide cap, or a salt thereof, is:

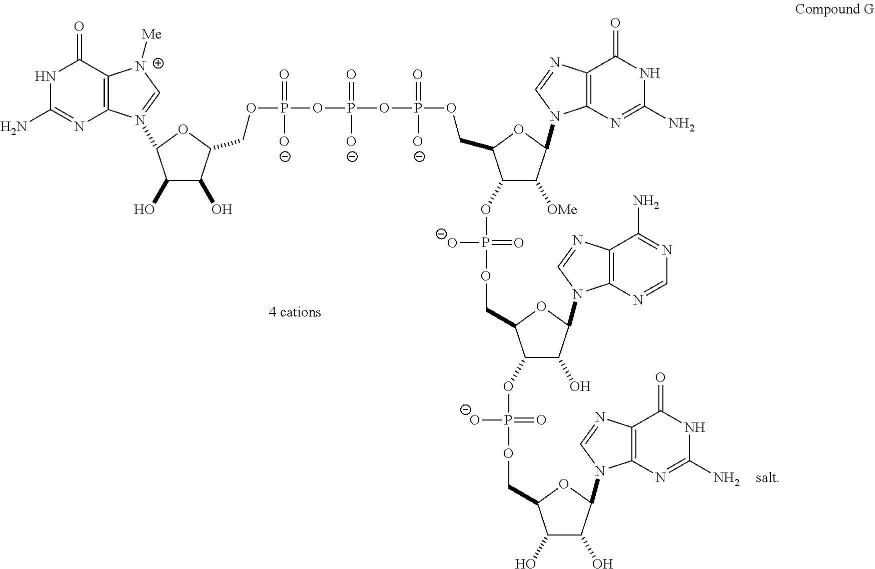

In some embodiments, the mRNA nucleotide cap, or a salt thereof, has a methylated guanosine and two or three nucleotides connected to a phosphate group. In some embodiments, the mRNA nucleotide cap is a salt. For example, one or more protons of the phosphate groups or other acidic positions of the nucleotide cap can be deprotonated, generating an anionic nucleotide cap. In some embodiments, the cation of the anionic nucleotide cap is an alkali metal ion (e.g., Li⁺, Na⁺, K⁺, Cs⁺ etc.). In some embodiments, the cation is Na⁺. In some embodiments, the cation of the anionic nucleotide cap is a primary, secondary, tertiary ammonium, or quaternary ammonium cation. In some embodiments, the cation is a primary ammonium cation. In some embodiments, the cation is ammonium. In some embodiments, the cation is an alkyl primary ammonium cation. In some embodiments, the alkyl primary ammonium cation is R₁H₃N wherein R₁ is C₁₋₈ alkyl. In some embodiments, the alkyl primary ammonium cation is methylammonium. In some embodiments, the cation is a secondary ammonium cation. In some embodiments, the cation is an alkyl secondary ammonium cation. In some embodiments, the alkyl secondary ammonium cation is (R₁)₂H₂N wherein each R₁ is independently C₁₋₈ alkyl. In some embodiments, the alkyl secondary ammonium cation is dimethylammonium or methylethylammonium. In some embodiments, the cation is a tertiary ammonium cation. In some embodiments, the cation is an alkyl tertiary ammonium cation. In some embodiments, the alkyl tertiary ammonium cation is (R₁)₃HN wherein each R₁ is independently C₁₋₈ alkyl. In some embodiments, the alkyl tertiary ammonium cation is dimethyloctylammonium, dimethylhexylammonium or triethylammonium. In some embodiments, the cation is a quaternary ammonium cation. In some embodiments, the cation is an alkyl quaternary ammonium cation. In some embodiments, the alkyl tertiary ammonium cation is (R₁)₄N wherein each R₁ is independently C₁₋₈ alkyl. In some embodiments, the alkyl quaternary ammonium cation is tetramethylammonium, trimethylethylammonium, or trimethylhexylammonium.

The anionic mRNA nucleotide cap can have one, two, three, four or more negative charges. In some embodiments, the anionic mRNA nucleotide cap has one negative charge. In some embodiments, the anionic mRNA nucleotide cap has two negative charges. In some embodiments, the anionic mRNA nucleotide cap has three negative charges. In some embodiments, the anionic mRNA nucleotide cap has four negative charges. The anionic mRNA nucleotide cap can have an average negative charge that is not limited to an integer, e.g., the average negative charge can be two and half, three and half, and four and half, etc. Salts of Compound A can include sodium salt (Na⁺), N,N-dimethyloctylammonium (DMOA) salt, dimethylhexylammonium (DMHA) salt, and primary ammonium (NH₄⁺) salt. Salts of Compound G can include sodium salt (Na⁺), DMOA salt, dimethylhexylammonium (DMHA) salt, and primary ammonium (NH₄⁺) salt.

The one or more mixtures that are collected and combined can be from an mRNA preparation. For example, the mRNA preparation is an in vitro transcription preparation. In some embodiments, the contaminant comprises proteins, macromolecules, nucleotide triphosphates (NTPs), side products, unused reagents, salts, or solvents.

The method provided herein includes removing the contaminants from the combined mixtures, which can include removing macromolecules and proteins from the combined mixture comprising the mRNA nucleotide cap, or a salt thereof, to provide a first mixture. The removal of the contaminants can further include concentrating and de salting the first mixture to provide a second mixture. In some embodiments, the removal of contaminants can further include removing nucleotide triphosphates (NTPs) and ion exchanging from the second mixture to provide a third mixture. In some embodiments, the removal of contaminants further includes concentrating and de-salting the third mixture to provide a fourth mixture. The removal of the contaminants can further include filtering, adjusting the concentration and pH of the fourth mixture. In some embodiments, adjusting the pH of the fourth mixture is optional. For example, the removal of contaminants from the combined mixtures comprises:
removing macromolecules and proteins from the combined mixture comprising the mRNA nucleotide cap, or a salt thereof, to provide a first mixture;
concentrating and de-salting the first mixture to provide a second mixture;
removing nucleotide triphosphates (NTPs) and ion exchanging from the second mixture to provide a third mixture; and
concentrating and de-salting the third mixture to provide a fourth mixture.

The removal of the contaminants from the combined mixture can include:
removing macromolecules and proteins from the combined mixture comprising the mRNA nucleotide cap, or a salt thereof, to provide a first mixture;
concentrating and de-salting the first mixture to provide a second mixture;
removing nucleotide triphosphates (NTPs) and ion exchanging from the second mixture to provide a third mixture;
concentrating and de-salting the third mixture to provide a fourth mixture; and filtering, and adjusting the concentration and pH of the fourth mixture.

The removal of the contaminants from the combined mixture can include:
removing macromolecules and proteins from the combined mixture comprising the mRNA nucleotide cap, or a salt thereof, to provide a first mixture;
concentrating and de-salting the first mixture to provide a second mixture;
removing nucleotide triphosphates (NTPs) and ion exchanging from the second mixture to provide a third mixture;
concentrating and de-salting the third mixture to provide a fourth mixture; and filtering and adjusting the concentration of the fourth mixture.

The removal of macromolecules and proteins from the combined mixtures comprising the mRNA nucleotide cap, or a salt thereof, to provide a first mixture can be carried out in various conditions. For example, the removing of macromolecules and proteins can include filtration. The filtration can be a pressure-driven membrane separation. In some embodiments, the filtration is tangential flow filtration. In some embodiments, the filtration system comprises a cassette filter, a spiral wound filter, a hollow fiber filter, a tubular filter, or a flat plate filter. In some embodiments, the filtration comprises a cassette filter or a spiral-wound filter. In some embodiments, the filtration comprises a filter selected from a cellulose based membrane, a polyamide membrane, a polyethersulfone membrane, a hydrophilic polyethersulfone membrane, a polyvinylidene fluoride membrane, and a polyethylene membrane. In some embodiments, the filtration comprises a cellulose based membrane filter. In some embodiments, the filtration comprises a polyamide thin film composite filter. In some embodiments, the filtration comprises a filter having a molecular weight cut off of about 1 kDa to about 100 kDa, about 3 kDa to about 50 kDa, about 5 kDa to about 20 kDa, or about 5 kDa to about 15 kDa. In some embodiments, the filtration comprises a filter having a molecular weight cut off of about 5 kDa or about 10 kDa.

The pH of the combined mixture comprising the mRNA nucleotide cap, or a salt thereof, can be adjusted before filtration. For example, the pH of the combined mixture comprising the mRNA nucleotide cap, or a salt thereof, can be adjusted to about 5.5 to about 7.0. In some embodiments, the pH is adjusted to about 6.0 to about 6.5.

In some embodiments, the pH of the combined mixture comprising the mRNA nucleotide cap, or a salt thereof, can be can be adjusted to about 8.0 or lower. In some embodiments, the pH is adjusted to about 4.0 to about 8.0. In some embodiments, the pH is adjusted to about 4.0, about 4.5, about 5.0, about 5.5, about 6.0, about 6.5, about 7.0, about 7.5, or about 8.0.

The removal of macromolecules can include removing RNA or pDNA. In some embodiments, the macromolecules removed are RNA.

In some embodiments, the macromolecules removed are mRNA.

The removal of macromolecules and proteins from the combined mixtures comprising the mRNA nucleotide cap, or a salt thereof, can be conducted between about 2 hours and about 6 hours. In some embodiments, the removing of macromolecules and proteins from the combined mixture comprising the mRNA nucleotide cap, or a salt thereof, to provide a first mixture is conducted in about 4 hours.

The removal of the contaminants can further comprise concentrating and de-salting the first mixture to provide the second mixture. The concentrating and de salting can be carried out under various conditions. In some embodiments, the first mixture is concentrated under vacuum. In some embodiments, the first mixture is concentrated at an elevated temperature. In some embodiments, the de-salting comprises filtration. In some embodiments, the filtration is a pressure-driven membrane separation. In some embodiments, the filtration is tangential flow filtration. In some embodiments, the filtration comprises a cassette filter, a spiral wound filter, a hollow fiber filter, a tubular filter, or a flat plate filter. In some embodiments, the filtration used to de-salt the first mixture comprises a cassette filter or a spiral-wound filter. In some embodiments, the filtration comprises a filter selected from a cellulose based membrane, a polyamide membrane, a polyethersulfone membrane, hydrophilic polyethersulfone membrane, polyvinylidene fluoride membrane, and a polyethylene membrane. In some embodiments, the filtration comprises a cellulose based membrane filter. In some embodiments, the filtration comprises a polyamide thin film composite filter. In some embodiments, the filtration comprises a filter having a molecular weight cut off of about 50 Da to about 5 kDa, about 100 Da to about 2 kDa, or 250 Da to about 2 kDa. In some embodiments, the filtration comprises a filter having a molecular weight cut off of about 300 Da to about 500 Da. In some embodiments, the filtration comprises a filter having a molecular weight cut off of about 600 Da to about 800 Da. In some embodiments, the filtration comprises a filter having a molecular weight cut off of about 2 kDa.

The concentration and de-salting of the first mixture to provide a second mixture can be conducted in about 1 hour to about 10 hours. In some embodiments, the concentrating and de-salting is conducted in about 3 hour to about 6 hours.

The removal of the contaminants can further comprises removing nucleotide triphosphates (NTPs) and ion exchanging from the second mixture to provide a third mixture. Removing the NTPs and ion exchanging can be carried out under various conditions. For example, the removing of NTPs can include passing the second mixture through an ion exchange chromatography system. The ion exchanging can include passing the second mixture through an ion exchange chromatography system. In some embodiments, the ion exchange chromatography system is an anion exchange system. In some embodiments, the anion exchange chromatography system comprises a mobile phase comprising water, an aqueous solution, or a buffered aqueous solution. In some embodiments, the mobile phase comprises an aqueous solution of NaCl, an aqueous solution of NH₄Cl, or an aqueous solution of KCl. In some embodiments, the mobile phase comprises water and an aqueous solution of NaCl. In some embodiments, the aqueous solution of NaCl has a concentration of about 0.20 M to about 2.0 M. In some embodiments, the aqueous solution of NaCl has a concentration of about 0.25 M to about 2.0 M. In some embodiments, the aqueous solution of NaCl has a concentration of about 0.5 M to about 2.0 M. In some embodiments, the aqueous solution of NaCl has a concentration of about 1.0 M to about 2.0 M. In some embodiments, the aqueous solution of NaCl has a concentration of about 1.5 M. In some embodiments, the mobile phase comprises water and an aqueous solution of NH₄Cl. In some embodiments, the aqueous solution of NH₄Cl has a concentration of about 0.5 M to about 1.5 M. In some embodiments, the aqueous solution of NH₄Cl has a concentration of about 1.0 M. In some embodiments, the anion exchange chromatography comprises exchanging N,N-dimethyloctylammonium (DMOA) for NH₄⁺. In some embodiments, the anion exchange chromatography system comprises a stationary phase comprising a strong base or a weak base. In some embodiments, the stationary phase comprises acrylic/divinylbenzene, styrene/divinylbenzene, hydroxylated methacrylic polymer, or crosslinked polymethacrylate. In some embodiments, the stationary phase comprises a functional group comprising dimethylamine, triethylamine, polyamine, a tertiary amine, a quaternary amine, dimethylethanolamine, or trimethylbenzylammonium. In some embodiments, the functional group comprises a quaternary amine. In some embodiments, the pump flow of the ion exchange chromatography system is about 50 mL/min to about 10 L/min, 65 mL/min to about 8 L/min, 75 mL/min to about 6 L/min, about 85 mL/min to about 1 L/min, or about 100 mL/min to about 800 mL/min. In some embodiments, the pump flow of the ion exchange chromatography system is about 110 mL/min, about 400 mL/min, or about 600 mL/min. In some embodiments, the pump flow of the ion exchange chromatography system is about 400 mL/min. In some embodiments, the pump flow of the ion exchange chromatography system is greater than about 10 L/min.

In some embodiments, the anion exchange chromatography comprises exchanging N,N-dimethyloctylammonium (DMOA) for NH₄⁺, K⁺, or Na⁺. In some embodiments, the stationary phase comprises TSKgel® SuperQ-5PW (20), TSKgel® SuperQ-5PW (30), TSKgel® SuperQ-650S, or POROS® XQ. TSKgel® SuperQ-5PW (20), TSKgel® SuperQ-5PW (30), TSKgel® SuperQ-650S, and POROS™ XQ can be purchased from Tosoh Bioscience, Inc. In some embodiments, the collection criteria is based on absorbance units (AU). In some embodiments, the collection criteria is about 100 to about 500 mAU, about 200 to about 400 mAU, about 250 to about 350 mAU, or about 300 mAU. In some embodiments, the mobile phase comprises an aqueous solution of KCl. In some embodiments, the aqueous solution of KCl has a concentration of about 0.20 M to about 2.0 M, about 0.25 M to about 2.0 M, about 0.5 M to about 2.0 M, about 0.8 M to about 1.5 M, or about 1.0 M.

In some embodiments, the ion exchange chromatography system comprises C₆H₈O₇²⁻, SO₄², PO₄², or Cl⁻. In some embodiments, the mobile phase comprises an aqueous solution comprising a cation selected from NH₄, K, and Na+ and an anion selected from C₆H₈O₇²⁻, SO₄², PO₄², and Cl⁻.

The removing of nucleotide triphosphates (NTPs) and ion exchanging the second mixture to provide a third mixture can be conducted between about 1 hours and about 10 hours or about 4 hours and about 9 hours. In some embodiments, removing of nucleotide triphosphates (NTPs) and ion exchanging the second mixture to provide a third mixture is conducted in about 9 hours. In some embodiments, the removing of nucleotide triphosphates (NTPs) and ion exchanging the second mixture to provide a third mixture is conducted between about 5 hours to about 20 hours, about 5 hours to about 15 hours, or 6 hours to about 10 hours. In some embodiments, the removing of nucleotide triphosphates (NTPs) and ion exchanging the second mixture to provide a third mixture is conducted in about 8 hours.

The removal of the contaminants can further comprise concentrating and de-salting the third mixture to provide a fourth mixture. The concentrating and de-salting can be carried out under various conditions. In some embodiments, the third mixture is concentrated under vacuum. In some embodiments, the third mixture is concentrated at an elevated temperature. In some embodiments, the de-salting of the third mixture comprises filtration. In some embodiments, the de-salting of the third mixture comprises passing the third mixture through a chromatography system. In some embodiments, the de-salting of the third mixture comprises passing the third mixture through a chromatography and filtration.

The chromatography system used for de-salting the third mixture can be, for example, reverse phase chromatography. In some embodiments, the reverse phase chromatography comprises a stationary phase comprising silica based, peptide based, or polymer based. In some embodiments, the stationary phase of the reverse phase chromatography comprises poly(styrene divinylbenzene) or C18 resin. In some embodiments, the stationary phase of the reverse phase chromatography comprises poly(styrene divinylbenzene). In some embodiments, the stationary phase of the reverse phase chromatography comprises C18 resin. In some embodiments, the stationary phase of the reverse phase chromatography is compatible with acetonitrile. In some embodiments, the reverse phase chromatography comprises a mobile phase comprising a polar solvent. In some embodiments, the mobile phase of the reverse phase chromatography is a buffer solution. In some embodiments, the mobile phase of the reverse phase chromatography is an ammonium salt buffer. In some embodiments, the mobile phase of the reverse phase chromatography is an alkyl ammonium salt buffer solution. In some embodiments, the mobile phase of the reverse phase chromatography is a dimethylhexylammonium (DMHA) buffer solution, a DMOA buffer solution, or a triethylammonium buffer solution. In some embodiments, the mobile phase of the reverse phase chromatography is a DMOA buffer solution. In some embodiments, the DMOA buffer solution the mobile phase of the reverse phase chromatography has a concentration of about 5 mM to about 15 mM. In some embodiments, the DMOA buffer solution of the mobile phase of the reverse phase chromatography has a concentration of about 10 mM. In some embodiments, the mobile phase of the reverse phase chromatography comprises an organic solvent. In some embodiments, the mobile phase of the reverse phase chromatography comprises a diol, an alcohol, an alkylhalide, an ether, a nitrile, or a mixture thereof. In some embodiments, the mobile phase of the reverse phase chromatography comprises a diol, a nitrile, or a mixture thereof. In some embodiments, the mobile phase of the reverse phase chromatography comprises hexylene glycol. In some embodiments, the mobile phase of the reverse phase chromatography comprises acetonitrile. In some embodiments, the reverse phase chromatography comprises a salt exchange. In some embodiments, the reverse phase chromatography comprises exchanging Na⁺ for N,N-dimethyloctylammonium (DMOA). In some embodiments, the pump flow of the reverse phase chromatography system is about 50 mL/min to about 10 L/min, 65 mL/min to about 8 L/min, 75 mL/min to about 6 L/min, about 85 mL/min to about 1 L/min, or about 100 mL/min to about 800 mL/min. In some embodiments, the pump flow of the reverse phase chromatography system is about 175 mL/min, about 400 mL/min, or about 600 mL/min. In some embodiments, the pump flow of the reverse phase chromatography system is about 175 mL/min. In some embodiments, the pump flow of the reverse phase chromatography system is greater than about 10 L/min.

The filtration used for de-salting the third mixture can be, for example, a pressure-driven membrane separation. In some embodiments, the filtration of the third mixture is tangential flow filtration. In some embodiments, the filtration of the third mixture comprises a cassette filter, a spiral wound filter, a hollow fiber filter, a tubular filter, or a flat plate filter. In some embodiments, the filtration of the third mixture comprises a cassette filter or a spiral-wound filter. In some embodiments, the filtration of the third mixture comprises a filter comprising a cellulose based membrane, a polyamide membrane, a polyethersulfone membrane, hydrophilic polyethersulfone membrane, polyvinylidene fluoride membrane, or a polyethylene membrane. In some embodiments, the filtration of the third mixture comprises a cellulose based membrane filter. In some embodiments, the filtration of the third mixture comprises a polyamide thin film composite filter. In some embodiments, the filtration comprises a filter having a molecular weight cut off of about 50 Da to about 5 kDa, about 100 Da to about 2 kDa, or 250 Da to about 1 kDa. In some embodiments, the filtration comprises a filter having a molecular weight cut off of about 300 Da to about 500 Da. In some embodiments, the filtration comprises a filter having a molecular weight cut off of about 600 Da to about 800 Da. In some embodiments, the filtration of the third mixture comprises a filter having a molecular weight cut off of about 2 kDa.

The passing of the third mixture through a chromatography system can be conducted in about 2 hours to about 10 hours. In some embodiments, the passing of the third mixture through a chromatography system is conducted in about 6 hours. The filtration of the third mixture can be conducted between about 2 hours and about 10 hours. In some embodiments, the filtration of the third mixture is conducted between about 3 hours and about 6 hours.

The removal of the contaminants can further comprise filtering, and adjusting the concentration and pH of the fourth mixture. The filtering, and adjusting the concentration and pH of the fourth mixture can be carried out under various conditions. For example, the filtering of the fourth mixture can comprise filtering the fourth mixture through a polyvinylidene filter, polyethylene filter, polypropylene filter, polytetrafluoroethylene filter, cellulose ester filter, or polyethersulfone filter. In some embodiments, the filtering of the fourth mixture comprises using a filter having a size of about a 0.1 μm to about 1 μm. In some embodiments, the filtering of the fourth mixture comprises using a filter having a size of about 0.2 μm. In some embodiments, the filtering of the fourth mixture comprises using a filter having a size of about 0.45 μm. In some embodiments, the concentration of the fourth mixture is adjusted to about 1000 mM to about 5 mM, about 500 mM to about 10 mM, about 250 mM to about 40 mM, or about 150 mM to about 50 mM. In some embodiments, the concentration of the fourth mixture is about 150 mM to about 75 mM or about 125 mM to about 85 mM. In some embodiments, the concentration of the fourth mixture is adjusted to about 100 mM. In some embodiments, the concentration of the fourth mixture is about 75 mM to about 25 mM or about 60 mM to about 40 mM. In some embodiments, the forth mixture is adjusted to about 50 mM, In some embodiments, the concentration of the fourth mixture is adjusted with a basic solution. In some embodiments, the basic solution used to adjust the concentration of the fourth mixture comprises NH₄OH, Na₂CO₃, NaHCO₃, K₂CO₃, KHCO₃, HClO, or CaCO₃. In some embodiments, the basic solution used to adjust the concentration of the fourth mixture comprises NH₄OH and water. In some embodiments, the basic solution used to adjust the concentration of the fourth mixture comprises about 1% w/v to about 8% w/v NH₄OH in water. In some embodiments, the basic solution used to adjust the concentration of the fourth mixture comprises about 3.5% w/v to 4.5% w/v NH₄OH in water. In some embodiments, the pH of the fourth mixture is adjust to about 5.5 to about 6.9. In some embodiments, the pH of the fourth mixture is adjust to about 6.0 to about 6.5. In some embodiments, the pH of the fourth mixture is adjust to about 6.3.

In some embodiments, the pH of the fourth mixture is adjusted to about 5.3 to about 7.3.

Alternatively, the removal of the contaminants from the combined mixtures can include:
removing nucleotide triphosphates (NTPs), macromolecules, and proteins from the combined mixture comprising the mRNA nucleotide cap, or a salt thereof, and ion exchanging the mixture to provide a first mixture;
concentrating and de-salting the first mixture to provide a second mixture;
concentrating, lyophilizing, reconstituting, and filtering the second mixture to provide the third mixture;
ion exchanging the third mixture to provide the fourth mixture;
concentrating, lyophilizing, reconstituting, concentrating, and filtering the fourth mixture to provide the fifth mixture; and
filtering, and adjusting the concentration and pH of the fifth mixture.

In the alternative method, the removal of the contaminants can comprises removing nucleotide triphosphates (NTPs), macromolecules, and proteins from the combined mixture comprising the mRNA nucleotide cap, or a salt thereof, and ion exchanging from the mixture to provide a first mixture. Removing the NTPs, macromolecules, and proteins and ion exchanging can be carried out under various conditions. For example, the removing of NTPs, macromolecules, and proteins can include passing the mixture through an ion exchange chromatography system. The ion exchanging can include passing the second mixture through an ion exchange chromatography system. In some embodiments, the ion exchange chromatography system is an anion exchange system. In some embodiments, the anion exchange chromatography system comprises a mobile phase comprising water, an aqueous solution, or a buffered aqueous solution. In some embodiments, the mobile phase comprises an aqueous solution of NaCl, an aqueous solution of NH₄Cl, or an aqueous solution of KCl. In some embodiments, the mobile phase comprises water and an aqueous solution of NaCl. In some embodiments, the aqueous solution of NaCl has a concentration of about 0.20 M to about 2.0 M. In some embodiments, the aqueous solution of NaCl has a concentration of about 0.25 M to about 2.0 M. In some embodiments, the aqueous solution of NaCl has a concentration of about 0.5 M to about 2.0 M. In some embodiments, the aqueous solution of NaCl has a concentration of about 1.0 M to about 2.0 M. In some embodiments, the aqueous solution of NaCl has a concentration of about 1.5 M. In some embodiments, the mobile phase comprises water and an aqueous solution of NH₄Cl. In some embodiments, the aqueous solution of NH₄Cl has a concentration of about 0.5 M to about 1.5 M. In some embodiments, the aqueous solution of NH₄Cl has a concentration of about 1.0 M. In some embodiments, the anion exchange chromatography comprises exchanging N,N-dimethyloctylammonium (DMOA) for NH₄⁺. In some embodiments, the anion exchange chromatography system comprises a stationary phase comprising a strong base or a weak base. In some embodiments, the stationary phase comprises acrylic/divinylbenzene, styrene/divinylbenzene, hydroxylated methacrylic polymer, or crosslinked polymethacrylate. In some embodiments, the stationary phase comprises a functional group comprising dimethylamine, triethylamine, polyamine, a tertiary amine, a quaternary amine, dimethylethanolamine, or trimethylbenzylammonium. In some embodiments, the functional group comprises a quaternary amine. In some embodiments, the pump flow of the ion exchange chromatography system is about 50 mL/min to about 10 L/min, 65 mL/min to about 8 L/min, 75 mL/min to about 6 L/min, about 85 mL/min to about 1 L/min, or about 100 mL/min to about 800 mL/min. In some embodiments, the pump flow of the ion exchange chromatography system is about 110 mL/min, about 400 mL/min, or about 600 mL/min. In some embodiments, the pump flow of the ion exchange chromatography system is about 110 mL/min.

In some embodiments, the anion exchange chromatography comprises exchanging N,N-dimethyloctylammonium (DMOA) for NH₄⁺, K⁺, or Na⁺. In some embodiments, the stationary phase comprises TSKgel® SuperQ-5PW (20), TSKgel® SuperQ-5PW (30), TSKgel® SuperQ-650S, or POROS™ XQ. In some embodiments, the collection criteria is based on absorbance units (AU). In some embodiments, the collection criteria is about 100 to about 500 mAU, about 200 to about 400 mAU, about 250 to about 350 mAU, or about 300 mAU. In some embodiments, the mobile phase comprises water and an aqueous solution of KCl. In some embodiments, the aqueous solution of KCl has a concentration of about 0.20 M to about 2.0 M, about 0.25 M to about 2.0 M, about 0.5 M to about 2.0 M, about 0.8 M to about 1.5 M, or about 1.0 M.

In some embodiments, the ion exchange chromatography system comprises C₆H₈O₇²⁻, SO₄², PO₄², or Cl⁻. In some embodiments, the mobile phase comprises an aqueous solution comprising a cation selected from NH₄, K, and Na⁺ and an anion selected from C₆H₈O₇²⁻, SO₄², PO₄², and Cl⁻.

In the alternative method, the removal of nucleotide triphosphates (NTPs), macromolecules, and proteins and ion exchanging the second mixture to provide a third mixture can be conducted between about 1 hours and about 10 hours or about 4 hours and about 9 hours. In some embodiments, removing of nucleotide triphosphates (NTPs), macromolecules, and proteins and ion exchanging the second mixture to provide a third mixture is conducted in about 9 hours. In some embodiments, the removing of nucleotide triphosphates (NTPs), macromolecules, and proteins and ion exchanging the second mixture to provide a third mixture is conducted between about 5 hours to about 20 hours, about 5 hours to about 15 hours, or 6 hours to about 10 hours. In some embodiments, the removing of nucleotide triphosphates (NTPs), macromolecules, and proteins and ion exchanging the second mixture to provide a third mixture is conducted in about 8 hours.

In the alternative method, the removal of the contaminants can further comprise concentrating and de-salting the first mixture to provide a second mixture. The concentrating and de-salting can be carried out under various conditions. In some embodiments, the first mixture is concentrated under vacuum. In some embodiments, the first mixture is concentrated at an elevated temperature. In some embodiments, the de salting of the first mixture comprises passing the first mixture through a chromatography system. In some embodiments, the chromatography system used for de-salting the first mixture comprises reverse phase chromatography. In some embodiments, the reverse phase chromatography comprises a stationary phase comprising silica based, peptide based, or polymer based. In some embodiments, the stationary phase of the reverse phase chromatography comprises poly(styrene divinylbenzene) or C18 resin. In some embodiments, the stationary phase of the reverse phase chromatography comprises poly(styrene divinylbenzene). In some embodiments, the stationary phase of the reverse phase chromatography comprises C18 resin. In some embodiments, the stationary phase of the reverse phase chromatography is compatible with acetonitrile. In some embodiments, the reverse phase chromatography comprises a mobile phase comprising a polar solvent. In some embodiments, the mobile phase of the reverse phase chromatography is a buffer solution. In some embodiments, the mobile phase of the reverse phase chromatography is an alkyl ammonium salt buffer solution. In some embodiments, the mobile phase of the reverse phase chromatography is a dimethylhexylammonium (DMHA) buffer solution, a DMOA buffer solution, or a triethylammonium buffer solution. In some embodiments, the mobile phase of the reverse phase chromatography is a DMOA buffer solution. In some embodiments, the DMOA buffer solution the mobile phase of the reverse phase chromatography has a concentration of about 5 mM to about 15 mM. In some embodiments, the DMOA buffer solution of the mobile phase of the reverse phase chromatography has a concentration of about 10 mM. In some embodiments, the mobile phase of the reverse phase chromatography comprises an organic solvent. In some embodiments, the mobile phase of the reverse phase chromatography comprises a diol, an alcohol, an alkylhalide, an ether, a nitrile, or a mixture thereof. In some embodiments, the mobile phase of the reverse phase chromatography comprises a diol, a nitrile, or a mixture thereof. In some embodiments, the mobile phase of the reverse phase chromatography comprises hexylene glycol. In some embodiments, the mobile phase of the reverse phase chromatography comprises acetonitrile.

The passing of the third mixture through a chromatography system can be conducted in about 2 hours to about 10 hours. In some embodiments, the passing of the third mixture through a chromatography system is conducted in about 6 hours.

In the alternative method, the removal of the contaminants can further comprise concentrating, lyophilizing, reconstituting, and filtering the second mixture to provide the third mixture. In some embodiments, the second mixture is concentrated under vacuum. In some embodiments, the second mixture is concentrated at elevated temperatures. In some embodiments, organic solvent is removed during concentration. In some embodiments, the organic solvent is acetonitrile. In some embodiments, lyophilizing removes water and DMOA. In some embodiments, the reconstituting is in an aqueous solvent. In some embodiments, the reconstituting is in water. In some embodiments, the reconstituting results in about a 100 mM to about a 300 mM solution. In some embodiments, the reconstituting results in about a 100 mM solution. In some embodiments, the second mixture is passed through a polyvinylidene filter, polyethylene filter, polypropylene filter, polytetrafluoroethylene filter, cellulose ester filter, or polyethersulfone filter. In some embodiments, the filter has a size of about 0.1 μm to about 1 μm. In some embodiments, the filter has a size of about 0.2 μm. In some embodiments, the filter has a size of about 0.45 μm.

The concentrating, lyophilizing, reconstituting, and filtering the second mixture to provide the third mixture can be conducted between about 30 hours to about 40 hours. In some embodiments, the concentrating, lyophilizing, reconstituting, and filtering the second mixture to provide the third mixture is conducted in about 36 hours.

In the alternative method, the removal of the contaminants can further comprise ion exchanging the third mixture to provide the fourth mixture. Ion exchanging the third mixture to provide the fourth mixture can be carried out under various conditions. For example, the ion exchanging comprises passing the third mixture through an ion exchange chromatography system. In some embodiments, the ion exchange chromatography system is an anion exchange system. In some embodiments, the mobile phase of the anion exchange chromatography system comprises water, an aqueous solution, or a buffered aqueous solution. In some embodiments, the mobile phase of the anion exchange chromatography system comprises an aqueous solution of NaCl, an aqueous solution of NH₄Cl, or an aqueous solution of KCl. In some embodiments, the mobile phase comprises water and an aqueous solution of NaCl. In some embodiments, the aqueous solution of NaCl has a concentration of about 0.20 M to about 2.0 M. In some embodiments, the aqueous solution of NaCl has a concentration of about 0.25 M to about 2.0 M. In some embodiments, the aqueous solution of NaCl has a concentration of about 0.5 M to about 2.0 M. In some embodiments, the aqueous solution of NaCl has a concentration of about 1.0 M to about 2.0 M. In some embodiments, the aqueous solution of NaCl has a concentration of about 1.5 M. In some embodiments, the mobile phase comprises water and an aqueous solution of NH₄Cl. In some embodiments, the aqueous solution of NH₄Cl has a concentration of about 0.5 M to about 1.5 M. In some embodiments, the aqueous solution of NH₄Cl has a concentration of about 1.0 M. In some embodiments, the anion exchange chromatography comprises exchanging DMOA for NH₄⁺. In some embodiments, the stationary phase of the anion exchange chromatography system comprises a strong base or a weak base. In some embodiments, the stationary phase of the anion exchange chromatography system comprises acrylic/divinylbenzene, styrene/divinylbenzene, hydroxylated methacrylic polymer, or crosslinked polymethacrylate. In some embodiments, the stationary phase of the anion exchange chromatography system has a functional group comprising a dimethylamine, triethylamine, polyamine, a tertiary amine, a quaternary amine, dimethylethanolamine, or trimethylbenzylammonium. In some embodiments, the stationary phase of the anion exchange chromatography system has a functional group comprising a trimethylbenzylammonium.

In some embodiments, the anion exchange chromatography comprises exchanging N,N-dimethyloctylammonium (DMOA) for NH₄⁺, K⁺, or Na⁺. In some embodiments, the stationary phase comprises TSKgel® SuperQ-5PW (20), TSKgel® SuperQ-5PW (30), TSKgel® SuperQ-650S, or POROS™ XQ. In some embodiments, the collection criteria is based on absorbance units (AU). In some embodiments, the collection criteria is about 100 to about 500 mAU, about 200 to about 400 mAU, about 250 to about 350 mAU, or about 300 mAU. In some embodiments, the mobile phase comprises an aqueous solution of KCl. In some embodiments, the aqueous solution of KCl has a concentration of about 0.20 M to about 2.0 M, about 0.25 M to about 2.0 M, about 0.5 M to about 2.0 M, about 0.8 M to about 1.5 M, or about 1.0 M.

In some embodiments, the ion exchange chromatography system comprises C₆H₈O₇⁻, SO₄², PO₄², or Cl⁻. In some embodiments, the mobile phase comprises an aqueous solution comprising a cation selected from NH₄, K, and Na⁺ and an anion selected from C₆H₈O₇²⁻, SO₄², PO₄², and Cl⁻.

In the alternative method, the removal of the contaminants can further comprise concentrating, lyophilizing, reconstituting, concentrating, and filtering the fourth mixture to provide the fifth mixture. In some embodiments, the second mixture is concentrated under vacuum. In some embodiments, the second mixture is concentrated at elevated temperatures. In some embodiments, organic solvent is removed during concentration. In some embodiments, the organic solvent is acetonitrile. In some embodiments, lyophilizing removes water and DMOA. In some embodiments, the reconstituting is in an aqueous solvent. In some embodiments, the reconstituting is in water. In some embodiments, the reconstituting results in about a 100 mM to about a 300 mM solution. In some embodiments, the reconstituting results in about a 100 mM solution. In some embodiments, the concentrating comprises cooling the fourth mixture, placing the fourth mixture under reduced vacuum, and heating the fourth mixture. In some embodiments, the fourth mixture is passed through a polyvinylidene filter, polyethylene filter, polypropylene filter, polytetrafluoroethylene filter, cellulose ester filter, or polyethersulfone filter. In some embodiments, the filter has a size of about 0.1 μm to about 1 μm. In some embodiments, the filter has a size of about 0.2 μm. In some embodiments, the filter has a size of about 0.45 μm.

The concentrating, lyophilizing, reconstituting, concentrating, and filtering the second mixture to provide the third mixture can be conducted between about 30 hours to about 40 hours. In some embodiments, the concentrating, lyophilizing, reconstituting, concentrating and filtering the second mixture to provide the third mixture is conducted in about 36 hours.

In the alternative method, the removal of the contaminants can further comprise filtering, and adjusting the concentration and pH of the fifth mixture. The filtering, and adjusting the concentration and pH of the fifth mixture can be carried out under various conditions. For example, the filtering of the fifth mixture can comprise filtering the fifth mixture through a polyvinylidene filter, polyethylene filter, polypropylene filter, polytetrafluoroethylene filter, cellulose ester filter, or polyethersulfone filter. In some embodiments, the filtering of the fifth mixture comprises using a filter having a size of about a 0.1 μm to about 1 μm. In some embodiments, the filtering of the fifth mixture comprises using a filter having a size of about 0.2 μm. In some embodiments, the filtering of the fifth mixture comprises using a filter having a size of about 0.45 μm. In some embodiments, the concentration of the fifth mixture is adjusted to about 1000 mM to about 5 mM, about 500 mM to about 10 mM, or about 250 mM to about 40 mM. In some embodiments, the concentration of the fifth mixture is adjusted to about 100 mM. In some embodiments, the fifth mixture is adjusted to about 50 mM. In some embodiments, the concentration of the fifth mixture is adjusted with a basic solution. In some embodiments, the basic solution used to adjust the concentration of the fifth mixture comprises NH₄OH, Na₂CO₃, NaHCO₃, K₂CO₃, KHCO₃, HClO, or CaCO₃. In some embodiments, the basic solution used to adjust the concentration of the fifth mixture comprises NH₄OH and water. In some embodiments, the basic solution used to adjust the concentration of the fifth mixture comprises about 1% w/v to about 8% w/v NH₄OH in water. In some embodiments, the basic solution used to adjust the concentration of the fifth mixture comprises about 3.5% w/v to 4.5% w/v NH₄OH in water. In some embodiments, the pH of the fifth mixture is adjust to about 5.5 to about 6.9. In some embodiments, the pH of the fifth mixture is adjust to about 6.0 to about 6.5. In some embodiments, the pH of the fifth mixture is adjust to about 6.3.

The recycle methods described herein can yield mRNA nucleotide caps, or salts thereof, with high purity, e.g., greater than about 80%. In some embodiments, the nucleotide cap, or a salt thereof, prepared by a method described herein has a purity greater than about 90%. In some embodiments, the nucleotide cap, or a salt thereof, prepared by a method described herein has a purity greater than about 95%. In some embodiments, the nucleotide cap, or a salt thereof, prepared by a method described herein has a purity greater than about 98%. In some embodiments, the nucleotide cap, or a salt thereof, prepared by a method described herein has a purity greater than about 99%. In some embodiments, the nucleotide cap, or a salt thereof, prepared by a method described herein has a purity greater than about 99.5%.

The methods described herein can also be applied to purification of mRNA nucleotide caps that were prepared in a synthetic reaction mixtures (e.g., de novo preparation). For example, the recycle methods described herein can include a process to remove the macromolecules and proteins from the mixture obtained from an mRNA preparation. The synthetic reaction to generate the mRNA cap may not contain contaminants like macromolecules and proteins, and as such, the process to remove macromolecules and proteins does not need to be performed.

Alternatively, the removal of the contaminants from the combined mixtures can include:
removing macromolecules from the combined mixture comprising the mRNA nucleotide cap, or a salt thereof, and adjusting the concentration to provide a first mixture;
removing proteins from the first mixture to provide a second mixture;
adjusting the concentration and filtering the second mixture to provide a third mixture;
removing nucleotide triphosphates (NTPs) and ion exchanging from the third mixture to provide a fourth mixture; and
adjusting the concentration and filtering the fourth mixture to provide a fifth mixture.

In the second alternative method, the removal of the contaminants can comprise removing macromolecules from the combined mixture comprising the mRNA nucleotide cap, or a salt thereof, and adjusting the concentration to provide a first mixture. The removal of macromolecules from the combined mixtures comprising the mRNA nucleotide cap, or a salt thereof, to provide a first mixture can be carried out in various conditions. For example, the removing of macromolecules can include filtration. The filtration can be a pressure-driven membrane separation. In some embodiments, the filtration is tangential flow filtration. In some embodiments, the filtration system comprises a cassette filter, a spiral wound filter, a hollow fiber filter, a tubular filter, or a flat plate filter. In some embodiments, the filtration comprises a cassette filter or a spiral-wound filter. In some embodiments, the filtration comprises a filter selected from a cellulose based membrane, a polyamide membrane, a polyethersulfone membrane, a hydrophilic polyethersulfone membrane, a polyvinylidene fluoride membrane, and a polyethylene membrane. In some embodiments, the filtration comprises a cellulose based membrane filter. In some embodiments, the filtration comprises a polyamide thin film composite filter. In some embodiments, the filtration comprises a filter having a molecular weight cut off of about 1 kDa to about 100 kDa, about 10 kDa to about 70 kDa, about 20 kDa to about 40 kDa, or about 25 kDa to about 35 kDa. In some embodiments, the filtration comprises a filter having a molecular weight cut off of about 30 kDa. In some embodiments the macromolecule is RNA, DNA, or mRNA.

In some embodiments, adjusting the concentration comprises adding an aqueous solution. In some embodiments, the aqueous solution is an acidic solution. In some embodiments, the acidic solution is a formic acid solution, an acetic acid solution, or a trichloroacetic acid solution. In some embodiments, the acidic solution is an acetic acid solution. In some embodiments, the aqueous solution has an acetic acid concentration of about 0.5 M to about 3 M, about 0.6 M to about 2 M, about 0.8 M to about 1.5 M, about 0.8 M to about 1.2 M, or about 1 M.

In the second alternative method, the removal of the contaminants can further comprise removing proteins from the first mixture to provide a second mixture. The removal of proteins from the combined mixtures comprising the mRNA nucleotide cap, or a salt thereof, to provide a first mixture can be carried out in various conditions. For example, the removing of proteins can include filtration. The filtration can be a pressure-driven membrane separation. In some embodiments, the filtration is tangential flow filtration. In some embodiments, the filtration system comprises a cassette filter, a spiral wound filter, a hollow fiber filter, a tubular filter, or a flat plate filter. In some embodiments, the filtration comprises a cassette filter or a spiral-wound filter. In some embodiments, the filtration comprises a filter selected from a cellulose based membrane, a polyamide membrane, a polyethersulfone membrane, a hydrophilic polyethersulfone membrane, a polyvinylidene fluoride membrane, and a polyethylene membrane. In some embodiments, the filtration comprises a cellulose based membrane filter. In some embodiments, the filtration comprises a polyamide thin film composite filter. In some embodiments, the filtration comprises a filter having a molecular weight cut off of about 1 kDa to about 100 kDa, about 3 kDa to about 50 kDa, about 5 kDa to about 20 kDa, or about 5 kDa to about 15 kDa. In some embodiments, the filtration comprises a filter having a molecular weight cut off of about 10 kDa.

In the second alternative method, the removal of the contaminants can further comprise adjusting the concentration and filtering the second mixture to provide a third mixture. The adjusting the concentration and filtering can be carried out under various conditions. In some embodiments, the concentration of the second mixture is adjusted to about 1000 mM to about 5 mM, about 500 mM to about 10 mM, or about 250 mM to about 40 mM. In some embodiments, the concentration of the second mixture is adjusted to about 100 mM. In some embodiments, the second mixture is adjusted to about 50 mM. In some embodiments, the concentration of the second mixture is adjusted with a basic solution. In some embodiments, the basic solution used to adjust the concentration of the fifth mixture comprises NH₄OH, Na₂CO₃, NaHCO₃, K₂CO₃, KHCO₃, HClO, or CaCO₃. In some embodiments, the basic solution used to adjust the concentration of the fifth mixture comprises NH₄OH and water. In some embodiments, the basic solution used to adjust the concentration of the second mixture comprises about 1% w/v to about 8% w/v NH₄OH in water. In some embodiments, the basic solution used to adjust the concentration of the second mixture comprises about 3.5% w/v to 4.5% w/v NH₄OH in water. In some embodiments, the filtration can be a pressure-driven membrane separation. In some embodiments, the filtration is tangential flow filtration. In some embodiments, the filtration system comprises a cassette filter, a spiral wound filter, a hollow fiber filter, a tubular filter, or a flat plate filter. In some embodiments, the filtration comprises a cassette filter or a spiral-wound filter. In some embodiments, the filtration comprises a filter selected from a cellulose based membrane, a polyamide membrane, a polyethersulfone membrane, a hydrophilic polyethersulfone membrane, a polyvinylidene fluoride membrane, and a polyethylene membrane. In some embodiments, the filtration comprises a cellulose based membrane filter. In some embodiments, the filtration comprises a polyamide thin film composite filter. In some embodiments, the filtration comprises a filter having a molecular weight cut off of about 0.5 kDa to about 30 kDa, about 0.7 kDa to about 20 kDa, about 0.8 kDa to about 10 kDa, or about 1 kDa to about 5 kDa. In some embodiments, the filtration comprises a filter having a molecular weight cut off of about 2 kDa.

In the second alternative method, the removal of the contaminants can further comprise removing nucleotide triphosphates (NTPs) and ion exchanging from the third mixture to provide a fourth mixture. Removing the NTPs and ion exchanging can be carried out under various conditions. For example, the removing of NTPs can include passing the second mixture through an ion exchange chromatography system. The ion exchanging can include passing the second mixture through an ion exchange chromatography system. In some embodiments, the ion exchange chromatography system is an anion exchange system. In some embodiments, the anion exchange chromatography system comprises a mobile phase comprising water, an aqueous solution, or a buffered aqueous solution. In some embodiments, the mobile phase comprises an aqueous solution of NaCl, an aqueous solution of NH₄Cl, or an aqueous solution of KCl. In some embodiments, the mobile phase comprises water and an aqueous solution of NaCl. In some embodiments, the aqueous solution of NaCl has a concentration of about 0.20 M to about 2.0 M. In some embodiments, the aqueous solution of NaCl has a concentration of about 0.25 M to about 2.0 M. In some embodiments, the aqueous solution of NaCl has a concentration of about 0.5 M to about 2.0 M. In some embodiments, the aqueous solution of NaCl has a concentration of about 1.0 M to about 2.0 M. In some embodiments, the aqueous solution of NaCl has a concentration of about 1.5 M. In some embodiments, the mobile phase comprises water and an aqueous solution of NH₄Cl. In some embodiments, the aqueous solution of NH₄Cl has a concentration of about 0.5 M to about 1.5 M. In some embodiments, the aqueous solution of NH₄Cl has a concentration of about 1.0 M. In some embodiments, the mobile phase comprises an aqueous solution of KCl. In some embodiments, the aqueous solution of KCl has a concentration of about 0.20 M to about 2.0 M, about 0.25 M to about 2.0 M, about 0.5 M to about 2.0 M, about 0.8 M to about 1.5 M, or about 1.0 M. In some embodiments, the anion exchange chromatography comprises exchanging N,N-dimethyloctylammonium (DMOA) for NH₄⁺, K⁺, or Na⁺. In some embodiments, the anion exchange chromatography comprises exchanging N,N-dimethyloctylammonium (DMOA) for NH₄⁺. In some embodiments, the anion exchange chromatography system comprises a stationary phase comprising a strong base or a weak base. In some embodiments, the stationary phase comprises acrylic/divinylbenzene, styrene/divinylbenzene, hydroxylated methacrylic polymer, or crosslinked polymethacrylate. In some embodiments, the stationary phase comprises a functional group comprising dimethylamine, triethylamine, polyamine, a tertiary amine, a quaternary amine, dimethylethanolamine, or trimethylbenzylammonium. In some embodiments, the functional group comprises a quaternary amine. In some embodiments, the stationary phase comprises TSKgel® SuperQ-5PW (20), TSKgel® SuperQ-5PW (30), TSKgel® SuperQ-650S, or POROS™ XQ. In some embodiments, the pump flow of the ion exchange chromatography system is about 50 mL/min to about 10 L/min, 65 mL/min to about 8 L/min, 75 mL/min to about 6 L/min, about 85 mL/min to about 1 L/min, or about 100 mL/min to about 800 mL/min. In some embodiments, the pump flow of the ion exchange chromatography system is about 110 mL/min, about 400 mL/min, or about 600 mL/min. In some embodiments, the pump flow of the ion exchange chromatography system is about 400 mL/min. In some embodiments, the pump flow of the ion exchange chromatography system is greater than about 10 L/min. In some embodiments, the collection criteria is based on absorbance units (AU). In some embodiments, the collection criteria is about 100 to about 500 mAU, about 200 to about 400 mAU, about 250 to about 350 mAU, or about 300 mAU.

In the second alternative method, the removal of the contaminants can further comprise adjusting the concentration and filtering the fourth mixture to provide a fifth mixture. The adjusting the concentration and filtering the fourth mixture to provide a fifth mixture can be carried out under various conditions. In some embodiments, the concentration of the fourth mixture is adjusted to about 1000 mM to about 5 mM, about 500 mM to about 10 mM, or about 250 mM to about 40 mM. In some embodiments, the concentration of the fourth mixture is adjusted to about 100 mM. In some embodiments, the fourth mixture is adjusted to about 50 mM. In some embodiments, the concentration of the fourth mixture is adjusted with a basic solution. In some embodiments, the basic solution used to adjust the concentration of the fourth mixture comprises NH₄OH, Na₂CO₃, NaHCO₃, K₂CO₃, KHCO₃, HClO, or CaCO₃. In some embodiments, the basic solution used to adjust the concentration of the fourth mixture comprises NH₄OH and water. In some embodiments, the basic solution used to adjust the concentration of the fourth mixture comprises about 1% w/v to about 8% w/v NH₄OH in water. In some embodiments, the basic solution used to adjust the concentration of the fourth mixture comprises about 3.5% w/v to 4.5% w/v NH₄OH in water. In some embodiments, the filtration can be a pressure-driven membrane separation. In some embodiments, the filtration is tangential flow filtration. In some embodiments, the filtration system comprises a cassette filter, a spiral wound filter, a hollow fiber filter, a tubular filter, or a flat plate filter. In some embodiments, the filtration comprises a cassette filter or a spiral-wound filter. In some embodiments, the filtration comprises a filter selected from a cellulose based membrane, a polyamide membrane, a polyethersulfone membrane, a hydrophilic polyethersulfone membrane, a polyvinylidene fluoride membrane, and a polyethylene membrane. In some embodiments, the filtration comprises a cellulose based membrane filter. In some embodiments, the filtration comprises a polyamide thin film composite filter. In some embodiments, the filtration comprises a filter having a molecular weight cut off of about 0.5 kDa to about 30 kDa, about 0.7 kDa to about 20 kDa, about 0.8 kDa to about 10 kDa, or about 1 kDa to about 5 kDa. In some embodiments, the filtration comprises a filter having a molecular weight cut off of about 2 kDa.

In some embodiments, the method of recycling the mRNA nucleotide cap can be conducted between about 1 day to about 20 days, about 2 days to about 15 days, about 3 days to about 10 days, about 5 days to about 10 days, about 6 days to about 8 days, or about 7 days. In some embodiments, the method of recycling the mRNA nucleotide cap results in about a 50% to about a 100% recovery, about a 60% to about a 100% recovery, about a 65% to about a 100% recovery, about a 70% to about 100% recovery, about a 70% to about a 99% recovery, or about a 70 to about a 85% recovery. In some embodiments, the mRNA nucleotide cap recovered from the method described herein has a purity of about 70% to about 100%, about 80% to about 100%, about 80% to about 99%, about 85% to about 99%, about 90% to about 99%, or about 95% to about 99%.

The cation associated with the mRNA nucleotide cap can change over the course of the recycling process. For example, during the recycling process the cation associated with the nucleotide cap before the recycling process can be exchanged to Na⁺, which is then exchanged to DMOA, which is then exchanged to NH₄⁺. In some embodiments, during the recycling process the cation associated with the nucleotide cap before the recycling process is exchanged to Na⁺, which is then exchanged to DMHA, which is then exchanged to NH₄⁺. In some embodiments, during the recycling process the cation associated with the nucleotide cap before the recycling process is exchanged to NH₄⁺, which is then exchanged to DMOA, which is then exchanged to NH₄⁺. In some embodiments, during the recycling process the cation associated with the nucleotide cap before the recycling process is exchanged to NH₄⁺, which is then exchanged to DMHA, which is then exchanged to NH₄⁺. In some embodiments, during the recycling process the cation associated with the nucleotide cap before the recycling process is exchanged to NH₄⁺, and the cation NH₄⁺ stays the same during the recycling process.

In some embodiments, the recycling process comprises:
filtering using tangential flow filtration (TFF), wherein the TFF comprises a filter with a molecular weight cut off of about 10 kDa to provide a first mixture;
filtering the first mixture using TFF, wherein the TFF comprises a filter with a molecular weight cut off of about 2 kDa to provide a second mixture;
performing anion exchange chromatography on the second mixture to provide a third mixture, wherein the anion exchange chromatography comprises exchanging the cation associated with the nucleotide cap with Na⁺ or NH₄⁺;
performing reverse phase chromatography on the third mixture to provide a fourth mixture, wherein the reverse phase chromatography comprises exchanging Na⁺ or NH₄⁺ for DMOA or DMHA; and
filtering the fourth mixture using TFF to provide a fifth mixture, wherein the filtering comprises a buffer exchange and swapping DMOA or DMHA for NH₄⁺.

In some embodiments, the recycling process comprises:
filtering using tangential flow filtration (TFF), wherein the TFF comprises a filter with a molecular weight cut off of about 2 kDa to provide a first mixture;
performing anion exchange chromatography on the first mixture to provide a second mixture, wherein the anion exchange chromatography comprises exchanging the cation associated with the nucleotide cap with NH₄; and
filtering the second mixture using TFF, wherein the TFF comprises a filter with a molecular weight cut off of about 2 kDa to provide a third mixture.

In order that the present disclosure can be more readily understood, certain terms are first defined. As used in this application, except as otherwise expressly provided herein, each of the following terms shall have the meaning set forth below. Additional definitions are set forth throughout the application.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values.

Nucleotides are referred to by their commonly accepted single-letter codes. Unless otherwise indicated, nucleic acids are written left to right in 5′ to 3′ orientation. Nucleobases are referred to herein by their commonly known one-letter symbols recommended by the IUPAC-IUB Biochemical Nomenclature Commission. Accordingly, A represents adenine, C represents cytosine, G represents guanine, T represents thymine, U represents uracil.

Alkyl: As used herein, the term “alkyl”, employed alone or in combination with other terms, refers to a saturated hydrocarbon group that may be straight-chain or branched. In some embodiments, the alkyl group contains 1 to 12, 1 to 8, or 1 to 6 carbon atoms. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, n-heptyl, n-octyl, and the like. In some embodiments, the alkyl moiety is methyl, ethyl, n-propyl, isopropyl, n-butyl, isobutyl, tert-butyl, n-pentyl, isopentyl, neopentyl, n-hexyl, or 2,4,4-trimethylpentyl. In some embodiments, the alkyl moiety is methyl.

About: The term “about” as used in connection with a numerical value throughout the specification and the claims denotes an interval of accuracy, familiar and acceptable to a person skilled in the art. Such interval of accuracy is ±10%.

Compound: As used herein, the term “compound,” is meant to include all stereoisomers and isotopes of the structure depicted. As used herein, the term “stereoisomer” means any geometric isomer (e.g., cis- and trans-isomer), enantiomer, or diastereomer of a compound. The present disclosure encompasses any and all stereoisomers of the compounds described herein, including stereomerically pure forms (e.g., geometrically pure, enantiomerically pure, or diastereomerically pure) and enantiomeric and stereoisomeric mixtures, e.g., racemates. Enantiomeric and stereomeric mixtures of compounds and means of resolving them into their component enantiomers or stereoisomers are well-known. “Isotopes” refers to atoms having the same atomic number but different mass numbers resulting from a different number of neutrons in the nuclei. For example, isotopes of hydrogen include tritium and deuterium. Further, a compound, salt, or complex of the present disclosure can be prepared in combination with solvent or water molecules to form solvates and hydrates by routine methods.

Diastereomer: As used herein, the term “diastereomer,” means stereoisomers that are not mirror images of one another and are non-superimposable on one another.

Enantiomer: As used herein, the term “enantiomer” means each individual optically active form of a compound of the present disclosure, having an optical purity or enantiomeric excess (as determined by methods standard in the art) of at least 80% (i.e., at least 90% of one enantiomer and at most 10% of the other enantiomer), at least 90%, or at least 98%.

Halo: As used herein, the terms “halo” and “halogen”, employed alone or in combination with other terms, refer to fluoro, chloro, bromo, and iodo.

In Vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

Isolated: As used herein, the term “isolated” refers to a substance or entity that has been separated from at least some of the components with which it was associated (whether in nature or in an experimental setting). Isolated substances (e.g., compounds) can have varying levels of purity in reference to the substances from which they have been isolated. Isolated substances and/or entities can be separated from at least about 10%, about 20%, about 30%, about 40%, about 50%, about 60%, about 70%, about 80%, about 90%, or more of the other components with which they were initially associated. In some embodiments, isolated substances are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

In some embodiments, the compounds described herein, and salts thereof, are substantially isolated. Methods for isolating compounds and their salts are routine in the art.

Substantially isolated: By “substantially isolated” is meant that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof.

Isomer: As used herein, the term “isomer” means any tautomer, stereoisomer, enantiomer, or diastereomer of any compound of the present disclosure. It is recognized that the compounds of the present disclosure can have one or more chiral centers and/or double bonds and, therefore, exist as stereoisomers, such as double-bond isomers (i.e., geometric E/Z isomers) or diastereomers (e.g., enantiomers (i.e., (+) or (−)) or cis/trans isomers). According to the present disclosure, the chemical structures depicted herein, and therefore the compounds of the present disclosure, encompass all of the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the present disclosure can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

Pharmaceutically acceptable: The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are used. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Pharmaceutically acceptable solvate: The term “pharmaceutically acceptable solvate,” as used herein, means a compound of the present disclosure wherein molecules of a suitable solvent are incorporated in the crystal lattice. A suitable solvent is physiologically tolerable at the dosage administered. For example, solvates can be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Purified: As used herein, “purify,” “purified,” “purification” means to make substantially pure or clear from unwanted components, material defilement, admixture or imperfection.

Salts: The term “salt” includes any anionic and cationic complex. Salts can include pharmaceutically acceptable salts. Non-limiting examples of anions include inorganic and organic anions, e.g., fluoride, chloride, bromide, iodide, oxalate (e.g., hemioxalate), phosphate, phosphonate, hydrogen phosphate, dihydrogen phosphate, oxide, carbonate, bicarbonate, nitrate, nitrite, nitride, bisulfite, sulfide, sulfite, bisulfate, sulfate, thiosulfate, hydrogen sulfate, borate, formate, acetate, benzoate, citrate, tartrate, lactate, acrylate, polyacrylate, fumarate, maleate, itaconate, glycolate, gluconate, malate, mandelate, tiglate, ascorbate, salicylate, polymethacrylate, perchlorate, chlorate, chlorite, hypochlorite, bromate, hypobromite, iodate, an alkylsulfonate, an arylsulfonate, arsenate, arsenite, chromate, dichromate, cyanide, cyanate, thiocyanate, hydroxide, peroxide, permanganate, and mixtures thereof.

Stereoisomer: As used herein, the term “stereoisomer” refers to all possible different isomeric as well as conformational forms that a compound can possess (e.g., a compound of any formula described herein), in particular all possible stereochemically and conformationally isomeric forms, all diastereomers, enantiomers and/or conformers of the basic molecular structure. Some compounds of the present disclosure can exist in different tautomeric forms, all of the latter being included within the scope of the present disclosure.

Substantially: As used herein, the term “substantially” refers to the qualitative condition of exhibiting total or near-total extent or degree of a characteristic or property of interest. One of ordinary skill in the biological arts will understand that biological and chemical characteristics rarely, if ever, go to completion and/or proceed to completeness or achieve or avoid an absolute result. The term “substantially” is therefore used herein to capture the potential lack of completeness inherent in many biological and chemical characteristics.

The methods described herein can be monitored according to any suitable method known in the art. For example, product formation can be monitored by spectroscopic means, such as nuclear magnetic resonance spectroscopy (e.g., ¹H or ¹³C), infrared spectroscopy, or spectrophotometry (e.g., UV-visible); or by chromatography such as high performance liquid chromatography (HPLC), liquid chromatography-mass spectrometry (LCMS or LC-MS), or thin layer chromatography (TLC) or other related techniques.

The methods described herein can be carried out in suitable solvents which can be readily selected by one of skill in the art of organic synthesis. Suitable solvents can be substantially nonreactive with the components (e.g., mRNA nucleotide caps) at the temperatures at which the processes are carried out, e.g., temperatures which can range from the solvent's freezing temperature to the solvent's boiling temperature. A given processes can be carried out in one solvent or a mixture of more than one solvent. Depending on the particular step, suitable solvents for a particular step can be selected. In some embodiments, methods described herein is to remove one or more solvents e.g., by heating, in vacuum such as rotavap.

Examples of solvents described herein can be an organic solvent, polar solvent, water, etc. or mixtures thereof. For example, the solvent can be a halogenated solvent, which can include carbon tetrachloride, bromodichloromethane, dibromochloromethane, bromoform, chloroform, bromochloromethane, dibromomethane, butyl chloride, dichloromethane, tetrachloroethylene, trichloroethylene, 1,1,1-trichloroethane, 1,1,2-trichloroethane, 1,1-dichloroethane, 2-chloropropane, α,α,α-trifluorotoluene, 1,2-dichloroethane, 1,2-dibromoethane, hexafluorobenzene, 1,2,4-trichlorobenzene, 1,2-dichlorobenzene, chlorobenzene, fluorobenzene, mixtures thereof and the like.

The solvent can be an organic solvent such as ether solvent, which can include dimethoxymethane, tetrahydrofuran, 1,3-dioxane, 1,4-dioxane, furan, diethyl ether, ethylene glycol dimethyl ether, ethylene glycol diethyl ether, diethylene glycol dimethyl ether, diethylene glycol diethyl ether, triethylene glycol dimethyl ether, anisole, t-butyl methyl ether, mixtures thereof and the like.

The solvent can be an organic solvent such as a hydrocarbon solvent, which can include benzene, cyclohexane, pentane, hexane, toluene, cycloheptane, methylcyclohexane, heptane (e.g., n-heptane), ethylbenzene, m-, o-, or p-xylene, octane, indane, nonane, naphthalene, mixtures thereof, and the like.

The solvent can be a polar solvent, which can be protic or aprotic solvent. Examples of protic solvents can include water, methanol, ethanol, 2-nitroethanol, 2-fluoroethanol, 2,2,2-trifluoroethanol, ethylene glycol, 1-propanol, 2-propanol, 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, glycerol, mixtures thereof, and the like.

Examples of aprotic solvents can include tetrahydrofuran (THF), N,N-dimethylformamide (DMF), N,N-dimethylacetamide (DMA), 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide, N-methylacetamide, N-methylformamide, acetonitrile, dimethyl sulfoxide, propionitrile, ethyl formate, methyl acetate, hexachloroacetone, acetone, ethyl methyl ketone, ethyl acetate, sulfolane, N,N-dimethylpropionamide, tetramethylurea, nitromethane, nitrobenzene, hexamethylphosphoramide, mixtures thereof, and the like.

In some embodiments, the compounds described herein, and salts thereof, can be found together with other substances such as water and solvents (e.g., hydrates and solvates).

The methods described herein can be carried out at appropriate temperatures, which can be readily determined by the skilled artisan. Temperatures will depend on, for example, the melting and boiling points of the components and solvent. “Elevated temperature” refers to temperatures above room temperature (about 22° C.). The expressions, “ambient temperature” and “room temperature” or “rt” as used herein, are understood in the art, and refer generally to a temperature, e.g., a reaction temperature, that is about the temperature of the room in which the method is carried out, for example, a temperature from about 20° C. to about 30° C.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

Section and table headings are not intended to be limiting.

### EXAMPLES

### Example 1

### Recycling Process for Compound A

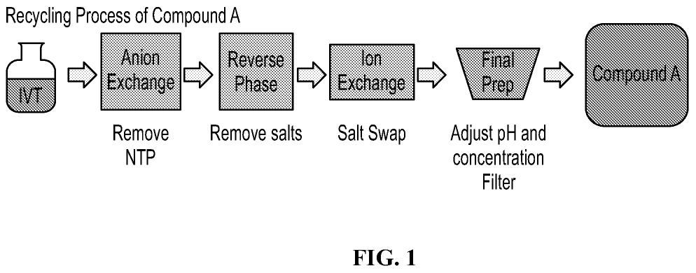

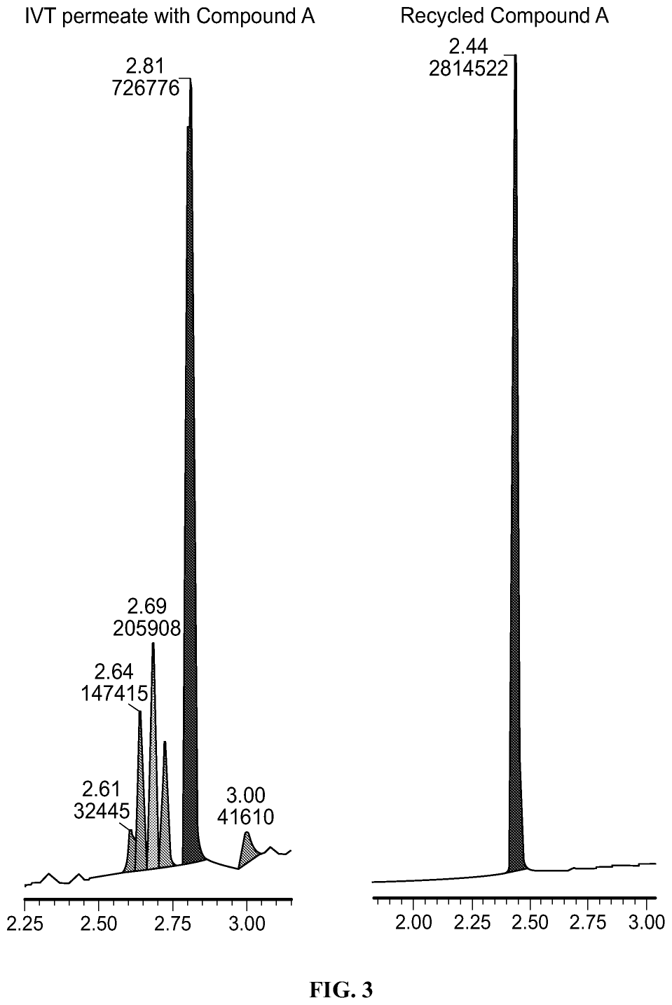

To remove the contaminants of mixtures containing unused/unreacted Compound A from in vitro transcription processes, these mixtures are combined and subjected to a series of purification processes. All material from a mixture comprising the mRNA nucleotide cap collected from an in vitro transcription preparation was converted to the Na⁺ salt and the nucleotide triphosphates (NTPs) and other contaminants from said mixture were removed via anion exchange chromatography. The solution was passed through a C18 column or a reverse phase polymer to convert to a DMOA⁺ salt and to undergo polishing purification. The resulting solution was concentrated via rotovap to remove acetonitrile, lyophilized to remove water and excess DMOA, reconstituted in water, and filtered. The solution was passed through an ion exchange resin to exchange the DMOA counter ion to NH₄. The solution was concentrated using a rotary evaporator, lyophilized, and reconstituted in water to 200 mM. The solution underwent freeze and thaw and then was filtered. The concentration of the solution was adjusted to around 100 mM, the pH was adjusted to 6.3, the concentration was adjusted to 100 mM, and the solution was filtered using a 0.2 μm filter resulting in purified Compound A (57.7 g, 66% —a sum over small runs). A schematic representation of the described process is shown in FIG. 1. FIG. 3 shows LCMS of the solution collected from in vitro transcription and of the purified Compound A.

### Example 2

### Alternative Recycling Process for Compound A

Another process for removing contaminants from a mixture containing Compound A is follows. The pH of a mixture comprising the mRNA nucleotide cap collected from an in vitro transcription preparation was adjusted to 6.0 to 6.5. The resulting solution filtered using tangential flow filtration (TFF) with a 5 kDa filter at room temperature to remove protein and macromolecules with the following parameters:
Pre-use sanitize membrane in 0.2%/pH 3-4, <25° C. peracetic acid recirc, 20 mins (<10 L)
Filter size=2519 13 sqft
Buffer type=IVT feed/water
Buffer volume=50-200 L
Feed flow rate=10-12 L/min
Permeate Flux range=0.75-1 L
TMP range=˜60 psig
Number of DV=1-3

The retentate was filtered using TFF with a 300-500 Da filter at room temperature with the following parameters:
Pre-use sanitize membrane in 0.2%/pH 3-4, <25° C. peracetic acid recirc, 20 mins (<10 L)
Filter size=2519, 13 sqft
Buffer=water
Buffer volume=70-220 L
Feed flow rate=10-12 L/min
Permeate Flux range=100-350 mL/min
TMP range=<150 psig
Initial concentration factor=5×
Number of DV=1-3

The permeate was monitored via LCMS for loss of Compound A. The retentate was concentrated and then was passed through a low pressure anion exchange chromatography system with UV detection (<5 bar) at room temperature to remove NTPs with the following parameters:
Standard pre-use sanitization
ÄKTA Pilot chromatography system
Chromatography column
   Bed volume=10 L or less
   Number of cycles=1 or more
Resin type=SuperQ®
Mobile Phase A=water
Mobile Phase B=1.5 M NaCl
Buffer volume A=100 L
Buffer volume B=50 L
Flow rate=0.4 LPM
Fraction collection volume=1 L
Total number fractions/cycle=40
Operational phases:
1 cv water
2 cv 5% 1.5M NaCl
3 cv 7.5% 1.5M NaCl
5.5 cv 11.5% 1.5M NaCl
0.5 cv 15% 1.5M NaCl
1 cv 100% 1.5M NaCl
2 cv water

IPC fraction analysis by LCMS was used to inform pooling and fraction rejection. The pooled fractions were passed through a medium pressure acetonitrile-compatible chromatography system with UV detection (<7 bar) at room temperature with the following parameters:
Standard pre-use sanitization
Chromatography column
Styrene divinylbenzene (Interchim 2×800 g Atoll-X®)
   Number of cycles=1 or more
Resin type=(Interchim Atoll X®) Styrene divinylbenzene
Mobile Phase A=10 mM DMOAB
Mobile Phase B=acetonitrile
Buffer volume A=40 L
Buffer volume B=20 L
Flow rate=0.175 LPM
Fraction collection volume=60 mL
Total number fractions/cycle=120-200
Operational phases:
1.5 CV 0% ACN, 100% 10 mM DMOAB
1.5 CV 5% ACN, 95% 10 mM DMOAB
1.5 CV 10% ACN, 90% 10 mM DMOAB
1.5 CV 15% ACN, 85% 10 mM DMOAB
1.0 CV 20% ACN, 80% 10 mM DMOAB
4.0 CV 22% ACN, 78% 10 mM DMOAB
0.5 CV 25% ACN, 75% 10 mM DMOAB
0.5 CV 30% ACN, 70% 10 mM DMOAB
1.0 CV 100% ACN, 0% 10 mM DMOAB
1.5 CV 0% ACN, 100% 10 mM DMOAB

Compound A eluted in ˜20% acetonitrile, and the column was regenerated using 100% acetonitrile. IPC fraction analysis by LCMS was used to inform pooling and fraction rejection. The pooled factions were concentrated under vacuum at 30° C. to remove acetonitrile and some DMOAB. The resulting mixture was filtered using TFF with a 300-500 Da filter at room temperature to exchange DMOA ions with NH₄ ions and de-salt with the following parameters:
Pre-use sanitize membrane in 0.2%/pH 3-4, <25° C. peracetic acid recirc, 20 mins (<10 L)
Filter size=2519 13 sqft
Buffer=400 mM NH₄C₁
Buffer volume=50 L
Feed flow rate=10-12 L/min
Permeate Flux range=200-400 mL/min
TMP range=<150 psig
Initial concentration factor=2-3×
Number of DV=8 w/400 mM NH₄Cl pH=6.3
Number of DV w/H₂O=5

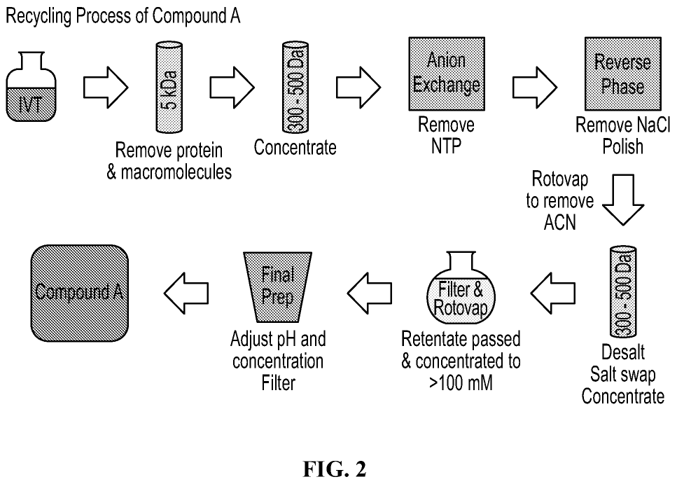

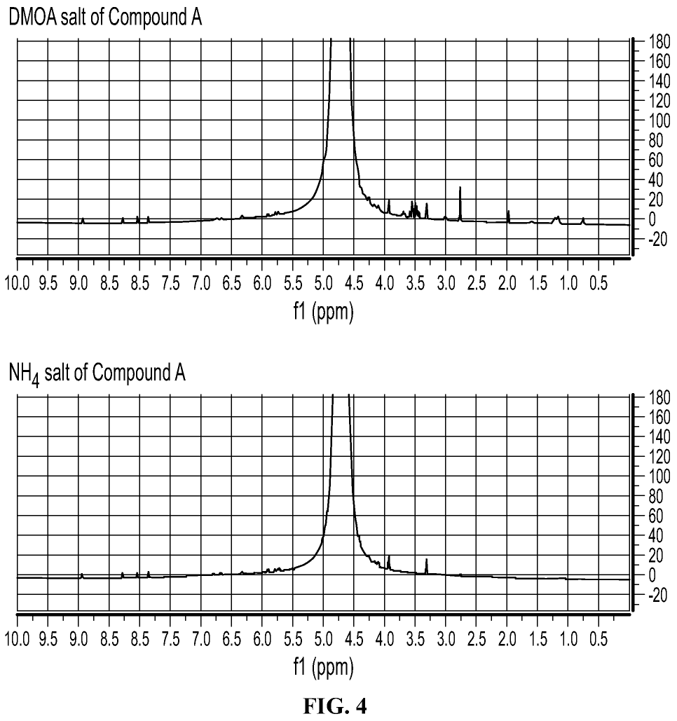

The completion of the salt swap was monitored via H¹ NMR. FIG. 4 shows the ¹H NMR of Compound A before and after ion exchange. The retentate was passed through a 0.2 μm or 0.45 μm filter and was then concentrated to >100 mM and to remove excess NH₄ under vacuum at 30° C. The concentration of the solution was adjusted to around 100 mM, the pH was adjusted to 6.3 using 4% NH₄OH in water, the concentration was adjusted to 100 mM, and the solution was filtered using a 0.2 μm filter resulting in purified Compound A (99.51% purity, about 80% recovery). A schematic representation of the described process is shown in FIG. 2.

The purity of recycled Compound A and de novo Compound A are shown in Table 1. De novo refers to Compound A purified from a reaction mixture obtained from the synthesis of the Compound A.

TABLE 1
Analytics and metal impurities of Compound A after purification.

| | | Analytics (by AD, %) | | | | | | |
|---|---|---|---|---|---|---|---|---|
| | Batch | Purity | dinucleotide* | +18 Imp | Metals by ICP-MS (ppm) | | | |
| | # | (%) | (%) | (%) | Zn | Na | Mg | Fe |
| Recycled | 1 | 99.51 | 0.03 | 0.25 | 152.6 | 58.4 | 48.5 | 4.1 |
| | 2 | 99.42 | 0.02 | 0.31 | 36.7 | 60.7 | 70 | 2.3 |
| | 3 | 99.28 | 0.03 | 0.29 | 8.5 | 45 | 11.9 | 0.6 |
| De novo | 4 | 97.26 | 0.17 | 1.11 | 4.3 | 7.4 | 18.1 | 4.1 |
| purification | 5 | 98.14 | 0.11 | 1.2 | 6.4 | 24.8 | 44.7 | 0.9 |
| | 6 | 99.17 | 0.08 | 0.5 | 28.7 | 27.1 | 38.2 | 1.2 |
| | 7 | 98.94 | 0.05 | 0.56 | 56.3 | 26 | 47.1 | 3.8 |
| | 8 | 99.05 | 0.07 | 0.56 | 29.1 | 14.6 | 18.1 | 7 |
| | 9 | 99.12 | 0.09 | 0.56 | 56.7 | 44.1 | 26.8 | 2.5 |
| | 10 | 98.94 | 0.14 | 0.55 | 94.5 | 61.6 | 124.6 | 6.8 |
| | 11 | 99.34 | 0.08 | 0.41 | 78.5 | 63.9 | 116.2 | 2.1 |

*dinucleotide refers to a nucleotide compound having two nucleotides.

### Example 3

### Recycling Process for Compound G

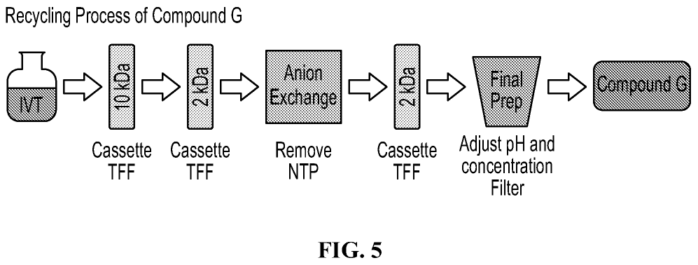

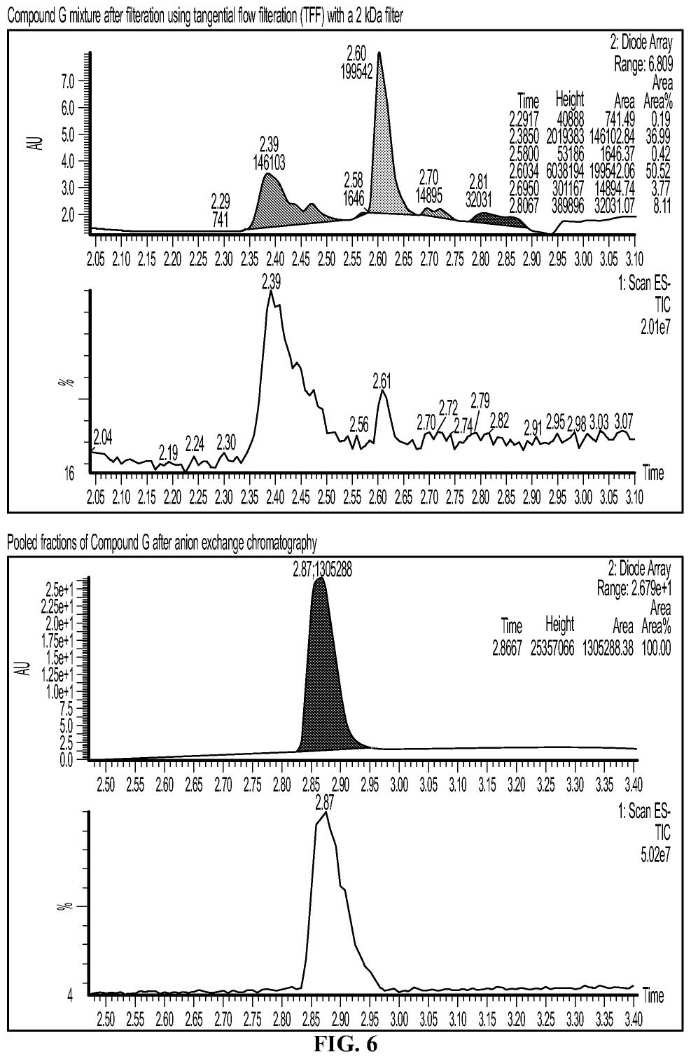

To remove the contaminants of mixtures containing unused/unreacted Compound G from in vitro transcription processes, these mixtures are combined and subjected to a series of purification processes. A mixture comprising the mRNA nucleotide cap collected from an in vitro transcription preparation was filtered using a cassette TFF with a 10 kDa filter to remove macromolecules and proteins. The resulting mixture concentrated and de-salted using a cassette TFF with a 2 kDa filter. LCMS of the resulting solution is shown in FIG. 6. The resulting solution underwent anion exchange chromatography using a SuperQ resin to remove reaction impurities and salt swap to NH₄⁺ (Buffer NH₄Cl). The process time was 2 days. On day 1 the column was prepped and sanitized for 4 hours. On day 2 the purification was performed over 8 hours, followed by fraction checking and pooling of fractions. LCMS of the pooled fractions is shown in FIG. 6. The resulting solution was concentrated and de-salted using a cassette TFF with a 2 kDa filter. The resulting solution was filtered through a 0.45 μm filter and concentrated via rotary evaporator as needed based on batch size for final concentration. The concentration of the solution was adjusted to around 50 mM, the pH was adjusted to 6.3 using 4% NH₄OH in water, the concentration was adjusted to 50 mM, and the solution was filtered using a 0.2 μm filter (about 90-95% purity, about 80-85% recovery). In some examples, the purity was greater than 95%. In some examples, the concentration of the solution was adjusted to less than 10 nM and the pH adjustment was not needed. A schematic representation of the described process is shown in FIG. 5.

Those skilled in the art will recognize, or be able to ascertain using no more than routine experimentation, many equivalents to the specific embodiments in accordance with the present disclosure described herein. The scope of the present disclosure is not intended to be limited to the above Description, but rather is as set forth in the appended claims.

In addition, it is to be understood that any particular embodiment of the present disclosure that falls within the prior art can be explicitly excluded from any one or more of the claims. Since such embodiments are deemed to be known to one of ordinary skill in the art, they can be excluded even if the exclusion is not set forth explicitly herein. Any particular embodiment of the compositions of the present disclosure can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

What is claimed is:

1. A method of recycling mRNA nucleotide cap or a salt thereof, from an mRNA preparation, comprising:
collecting and combining one or more mixtures comprising the nucleotide mRNA cap, or a salt thereof, and one or more contaminants; and
removing the contaminants from the combined mixtures.

2. The method of claim 1, wherein the mRNA nucleotide cap, or a salt thereof, is:

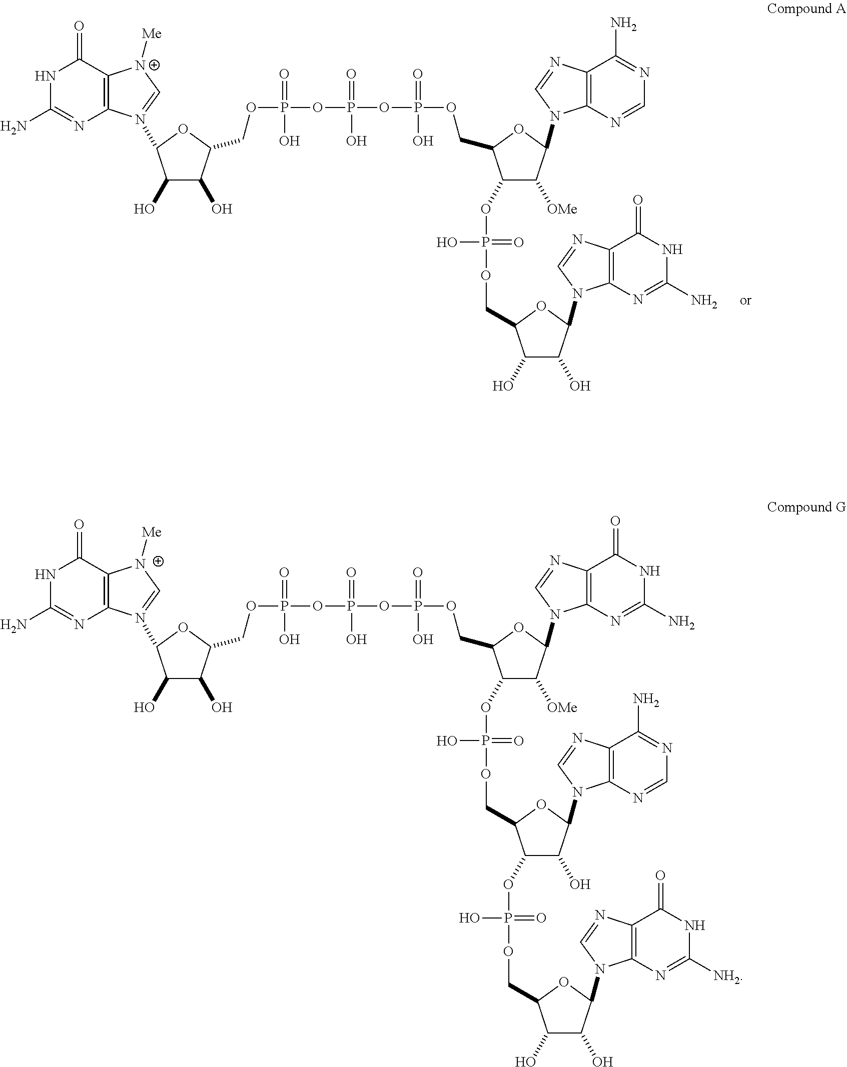

3. The method of claim 1, wherein the mRNA nucleotide cap, or a salt thereof, is:

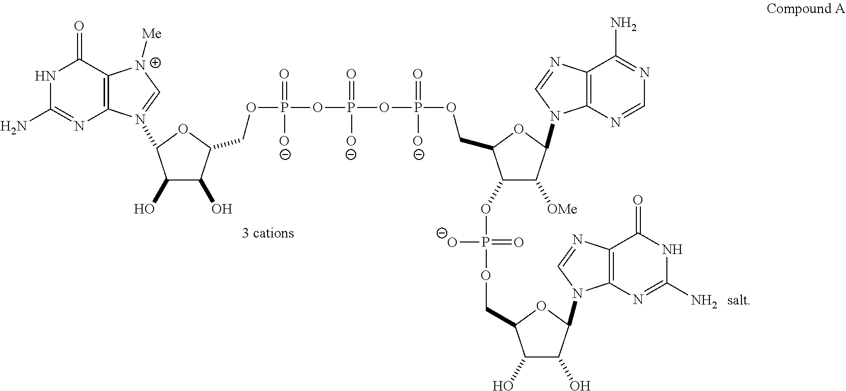

4. The method of claim 1, wherein the mRNA nucleotide cap, or a salt thereof, is:

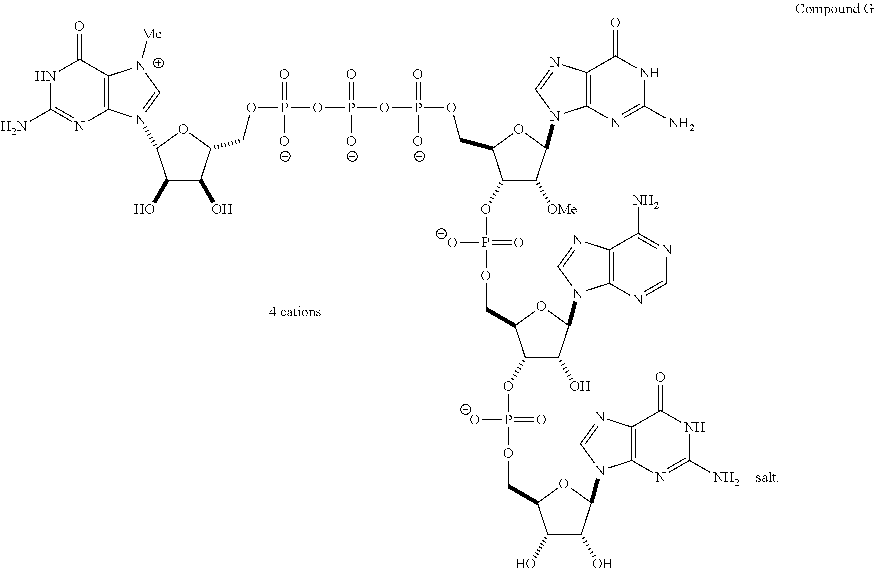

5. The method of claim 3 or 4, wherein the cation is ammonium.

6. The method of claim 3 or 4, wherein the cation is dimethyloctylammonium, dimethylhexylammonium or triethylammonium.

7. The method of any one of claims 1-6, wherein the removing comprises:
removing macromolecules and proteins from the combined mixture comprising the mRNA nucleotide cap, or a salt thereof, to provide a first mixture.

8. The method of claim 7, wherein the removing of macromolecules and proteins comprises filtration.

9. The method of claim 8, wherein the filtration used to remove macromolecules and proteins is tangential flow filtration.

10. The method of claim 8 or 9, wherein the filtration used to remove macromolecules and proteins comprises a cassette filter or spiral-wound filter.

11. The method of any one of claims 8-10, wherein the filtration used to remove macromolecules and proteins comprises a cellulose based membrane filter.

12. The method of any one claims 8-10, wherein the filtration used to remove macromolecules and proteins comprises a polyamide thin film composite filter.

13. The method of any one of claims 8-12, wherein the filtration used to remove macromolecules and proteins comprises a filter having a molecular weight cut off of about 5 kDa to about 15 kDa.

14. The method of any one of claims 7-13, wherein the pH is adjusted to about 6.0 to about 6.5.

15. The method of any one of claims 1-6, wherein the removing further comprises:
concentrating and de-salting the first mixture to provide a second mixture.

16. The method of claim 15, wherein the de-salting comprises filtration.

17. The method of claim 16, wherein the filtration used to de-salt the first mixture is tangential flow filtration.

18. The method of claim 16 or 17, wherein the filtration used to de-salt the first mixture comprises a cassette filter or spiral-wound filter.

19. The method of any one of claims 16-18, wherein the filtration used to de-salt the first mixture comprises a cellulose based membrane filter.

20. The method of any one of claims 16-18, wherein the filtration used to de-salt the first mixture comprises a polyamide thin film composite filter.

21. The method of any one of claims 16-20, wherein the filtration used to de-salt the first mixture comprises a filter having a molecular weight cut off of about 50 Da to about kDa.

22. The method of any one of claims 16-20, wherein the filtration used to de-salt the first mixture comprises a filter having a molecular weight cut off of about 2 kDa.

23. The method of any one of claims 1-6, wherein the removing further comprises:
removing nucleotide triphosphates (NTPs) and ion exchanging from the second mixture to provide a third mixture.

24. The method of claim 23, wherein the removing of NTPs comprises passing the second mixture through an ion exchange chromatography system.

25. The method of claim 23 or 24, wherein the ion exchange chromatography system is an anion exchange system.

26. The method of claim 25, wherein the anion exchange chromatography system comprises a mobile phase comprising water, an aqueous solution, or a buffered aqueous solution.

27. The method of claim 25 or 26, wherein the mobile phase comprises water and an aqueous solution of NaCl.

28. The method of claim 27, wherein the aqueous solution of NaCl has a concentration of about 0.25 M to about 2.0 M.

29. The method of claim 25 or 26, wherein the mobile phase comprises water and an aqueous solution of NH₄Cl.

30. The method of claim 29, wherein the aqueous solution of NH₄Cl has a concentration of about 0.5 M to about 1.5 M.

31. The method of any one of claims 25-30, wherein the anion exchange chromatography comprises exchanging N,N-dimethyloctylammonium (DMOA) for NH₄⁺.

32. The method of any one of claims 25-31, wherein the anion exchange chromatography system comprises a stationary phase comprising a strong base or a weak base.

33. The method of claim 32, wherein the stationary phase comprises a functional group comprising dimethylamine, triethylamine, polyamine, a tertiary amine, a quaternary amine, dimethylethanolamine, or trimethylbenzylammonium.

34. The method of any one of claims 24-33, wherein the pump flow of the ion exchange chromatography system is about 50 mL/min to about 10 L/min.

35. The method of any one of claims 1-6, wherein the removing further comprises:
concentrating and de-salting the third mixture to provide a fourth mixture.

36. The method of claim 35, wherein the de-salting of the third mixture comprises passing the third mixture through a chromatography system or filtration, or combination thereof.

37. The method of claim 36, wherein the chromatography system used for de-salting the third mixture is a reverse phase chromatography.

38. The method of claim 37, wherein the reverse phase chromatography comprises a stationary phase comprising poly(styrene divinylbenzene) or C18 resin.

39. The method of claim 37 or 38, wherein the reverse phase chromatography comprises a mobile phase comprising a DMOA buffer solution.

40. The method of claim 39, wherein the DMOA buffer solution of the mobile phase of the reverse phase chromatography has a concentration of about 5 mM to about 15 mM.

41. The method of claim 37 or 38, wherein the reverse phase chromatography comprises a mobile phase comprising acetonitrile.

42. The method of any one of claims 37-41, wherein the reverse phase chromatopgrahy further comprises a salt exchange.

43. The method of any one of claims 37-42, wherein the reverse phase chromatography comprises exchanging Na⁺ for N,N-dimethyloctylammonium (DMOA).

44. The method of any one of claims 37-43, wherein the pump flow of the reverse phase chromatography system is about 50 mL/min to about 10 L/min.

45. The method of claim 36, wherein the filtration of the third mixture is tangential flow filtration.

46. The method of claim 36 or 45, wherein the filtration of the third mixture comprises a cassette filter or spiral-wound filter.

47. The method of any one of claims 36, 45, and 46, wherein the filtration of the third mixture comprises a cellulose based membrane filter.

48. The method of any one of claims 36, 45, and 46, wherein the filtration of the third mixture comprises a polyamide thin film composite filter.

49. The method of any one of claims 36 and 45-48, wherein the filtration of the third mixture comprises a filter having a molecular weight cut off of about 50 Da to about 5 kDa.

50. The method of any one of claims 1-6, wherein the removing further comprises:
filtering, and adjusting the concentration and pH of the fourth mixture.

51. The method of claim 50 comprising filtering the fourth mixture through a polyvinylidene filter, polyethylene filter, polypropylene filter, polytetrafluoroethylene filter, cellulose ester filter, or polyethersulfone filter.

52. The method of claim 50 or 51, wherein the filtering of the fourth mixture comprises using a filter having a size of about a 0.1 μm to about 1 μm.

53. The method of claim 50, wherein the concentration of the fourth mixture is adjusted to about 1000 mM to about 25 mM.

54. The method of claim 50, wherein the concentration of the fourth mixture is adjusted to about 100 mM.

55. The method of claim 50, wherein the concentration of the fourth mixture is adjusted to about 50 mM.

56. The method of any one of claims 50 and 53-55, wherein the concentration of the fourth mixture is adjusted with a basic solution.

57. The method of claim 56, wherein the basic solution used to adjust the concentration of the fourth mixture comprises NH₄OH and water.

58. The method of claim 56, wherein the basic solution used to adjust the concentration of the fourth mixture comprises about 3.5% w/v to 4.5% w/v NH₄OH in water.

59. The method of any one of claims 50 and 53-58, wherein the pH of the fourth mixture is adjust to about 5.3 to about 7.3.
